(19) United States
(12) Patent Application Publication
(10) Pub. No.: US 2013/0028963 A1
(43) Pub. Date: Jan. 31, 2013

(54) Forming an Artificial Cell with controlled membrane composition, asymmetry, and contents

(71) Applicants: Daniel A. Fletcher, Berkeley, CA (US); Thomas Li, Modesto, CA (US); Sapun Parekh, Washington, DC (US); Jeanne Stachowiak, Oakland, CA (US); Allen Liu, San Diego, CA (US); David Richmond, Berkeley, CA (US); Eva Schmid, San Francisco, CA (US)

(72) Inventors: Daniel A. Fletcher, Berkeley, CA (US); Thomas Li, Modesto, CA (US); Sapun Parekh, Washington, DC (US); Jeanne Stachowiak, Oakland, CA (US); Allen Liu, San Diego, CA (US); David Richmond, Berkeley, CA (US); Eva Schmid, San Francisco, CA (US)

(73) Assignee: THE REGENTS OF THE UNIVERSITY OF CALIFORNIA, Oakland, CA (US)

(21) Appl. No.: 13/631,087

(22) Filed: Sep. 28, 2012

Related U.S. Application Data
(63) Continuation of application No. PCT/US2011/030509, filed on Mar. 30, 2011.
(60) Provisional application No. 61/320,232, filed on Apr. 1, 2010.

(51) Int. Cl. A61K 9/127; A61J 3/00; A61K 38/00; A61K 31/7088; A61K 9/16; A61K 38/43

(52) U.S. Cl. 424/450; 424/491; 424/94.1; 514/1.1; 514/44 R; 264/4

(57) ABSTRACT

The present invention provides a vesicle having a unilamellar bilayer including a lipid and a second bilayer component selected from a membrane protein or a functionalized lipid. The vesicle also includes a component encapsulated by the unilamellar bilayer, wherein the encapsulated component includes a protein, a peptide, an enzyme, an oligonucleotide, or a polynucleotide. Also included are methods of making the vesicles of the present invention.

### CROSS-REFERENCES TO RELATED APPLICATIONS

This application is a continuation application of International Patent Application No. PCT/US2011/030509, filed on Mar. 30, 2011, which application claims priority to U.S. Provisional Application No. 61/320,232, filed Apr. 1, 2010, all of which are incorporated by reference in their entirety herein for all purposes.

### STATEMENT AS TO RIGHTS TO INVENTIONS MADE UNDER FEDERALLY SPONSORED RESEARCH OR DEVELOPMENT

This work was supported by the Nanomedicine Development Center Grant No. 10986-31150-44-IQDAF awarded by the National Institutes of Health, and under Contract No. DE-AC02-05CH11231 awarded by the U.S. Department of Energy. The government has certain rights in this invention.

### BACKGROUND OF THE INVENTION

Lipid bilayer membranes provide the archetypal organizing structure by which cells separate themselves from their environment and internally compartmentalize and transport molecules. At the molecular level, cellular membranes are a crowded mix of many different lipids and proteins, and their composition and organization are crucial to a broad range of cellular functions. In endo- and exocytosis, apoptosis, signal transduction and motility, membranes serve as substrates for the activity of specialized lipids, transmembrane proteins, and associated binding proteins. Moreover, cells use cycles of endo- and exocytosis to dynamically regulate cell membrane composition and area.

Encapsulation of enzymes in lipid vesicles was first attempted by Sessa and Weissmann in 1970 (Sessa & Weissman.(1970) Journal of Biological Chemistry 245 :3295). Since then, vesicles encapsulating biologically-active compounds have been used as chemical micro-reactors (Walde (1996) Current Opinion in Colloid & Interface Science 1:638), delivery vehicles for pharmaceuticals (Lasic & Papahadjopoulos (1995) Liposomes Science 267: 1275), and platforms for synthetic biological systems (Chen, et al. (2004) Science 305: 1474). In order to identify the functional role of membrane composition and organization in cells, traditional cell biological approaches are increasingly being complemented by in vitro experiments aimed at reconstituting cellular behavior from a minimal system of components. For example, synthetic lipid vesicles have been used to study the necessary and sufficient protein machinery for membrane fusion, membrane deformation by cytoskeletal proteins, and scission by membrane binding proteins.

Since mixtures of lipids and biomolecules will not spontaneously organize into solution-encapsulating vesicles of defined composition and size, several methods have been devised to form and load vesicles including swelling (Reeves & Dowben (1969) Journal of Cellular Physiology 73:49), extrusion (Olson, et al. (1979) Biochimica Et Biophysica Acta 557:9), electro formation (Angelova & Dimitrov (1986) Faraday Discussions 81:303), electroinjection (Karlsson, et al. (2000) Analytical Chemistry 72:5857), and reverse evaporation and emulsion (Szoka & Papahadjopoulos (1978) Proceedings of the National Academy of Sciences of the United States of America 75:4194; Pautot, et al. (2003) Proceedings of the National Academy of Sciences of the United States of America 100: 10718). Among the most important properties of a vesicle formation and loading technique are (i) control of membrane unilamellarity, (ii) control of vesicle size, and (iii) control of internal solution concentration without solute-specific selectivity. Furthermore, practical applications of vesicle encapsulation require high encapsulation efficiency to minimize needed solution volume, highthroughput formation, and the ability to examine the vesicle and any associated reactions immediately after loading. Furthermore, in applications to study in vitro protein assemblies (Liu, A. P. & Fletcher, D. A. (2006) Actin Polymerization Serves as a Membrane Domain Switch in Model Lipid Bilayers. Biophys. J 91 :4064-4070) and cell-like membrane deformations (Miyata, H., Nishiyama, S., Akashi, K. -i. & Kinosita, K., Jr. (1999) Protrusive growth from giant liposomes driven by actin polymerization. Proceedings of the National Academy of Sciences 96:2048-2053), giant unilamellar vesicles (GUVs) with diameters >10 μm are desirable to facilitate direct visualization of internal behavior by light microscopy.

Though each existing vesicle formation technique achieves some of these criteria, none enables vesicle formation and encapsulation with all of these properties. For example, swelling typically results in the formation of multilamellar vesicles (MLV s) that vary widely in size and encapsulate with low, solute-specific efficiency. Electroformation can produce giant unilamellar vesicles (GUVs) with diameters above 10 μm. However, vesicle diameter is not controlled, and the technique is restricted to low ionic strength conditions, limiting its applicability for encapsulation of biomolecules (Bucher, P., Fischer, A., Luisi, P. L., Oberholzer, T. & Walde, P. (1998) Giant vesicles as biochemical compartments: The use of micro injection techniques. Langmuir 14:2712-2721. 26). The size uniformity of GUVs and the unilamellarity of MLVs made by several techniques can be greatly enhanced by extruding the vesicles through filters with sub-micron pores, though the resulting vesicles are limited to the size of the pores and have the same internal composition as the original vesicles (Colletier, J. -P., Chaize, B., Winterhalter, M. & Fournier, D. (2002) Protein encapsulation in liposomes: efficiency depends on interactions between protein and phospholipid bilayer. BMC Biotechnology 2:9). The reverse emulsion technique, offers the advantage of soluteindependent encapsulation efficiency. However the size of vesicles produced by reverse emulsion is not directly controllable and throughput is limited (Pautot, S., Frisken, B. J. & Weitz, D. A. (2003) Production of unilamellar vesicles using an inverted emulsion. Langmuir 19:2870-2879). Recently, several groups have reported the high-throughput production of monodisperse single and double emulsion structures using microdevices that hydrodynamically focus fluid streams (Atencia, J. & Beebe, D. J. (2005) Controlled micro fluidic interfaces. Nature 437:648-655; Utada, A. S., Lorenceau, E., Link, D. R., Kaplan, P. D., Stone, H. A. & Weitz, D. A. (2005) Monodisperse double emulsions generated from a microcapillary device. Science 308:537-541; Gunther, A. & Jensen, K. F. (2006) Multiphase microfluidics: from flow characteristics to chemical and materials synthesis. Lab on a Chip 6: 1487-1503) and pulsed microfluidic jets that deform interfaces (Funakoshi, K., Suzuki, H. & Takeuchi, S. (2007) Formation of Giant Lipid Vesicle-like Compartments from a Planar Lipid Membrane by a Pulsed Jet Flow. Journal of the American Chemical Society), though none have been shown to form unilamellar vesicles, an essential requirement for many applications. In a previous report, we showed that it was possible to use a precisely-controlled micro fluidic jet propelled by a piezo-actuated syringe to deform a lipid bilayer membrane into a giant unilamellar vesicle capable of incorporating membrane proteins. l. C. Stachowiak, D. L. Richmond, T. H. Li, A. P. Liu, S. H. Parekh and D. A. Fletcher, Proc Natl Acad Sci, 2008, 105, 4697-4702. While this system successfully demonstrated the formation ofunilamellar vesicles by microfluidic jetting, it was severely limited in the throughput of vesicle formation and did not achieve variation of vesicle size or production of cell-sized vesicles.

In order to capture the essential features of complex cellular processes by reconstitution, methods are required to assemble purified components in ways that more faithfully emulate real cells. For example, properties that are believed to influence membrane processes and are therefore desirable to control in reconstitutions include asymmetric lipid composition, insertion of membrane proteins, physical properties such as membrane tension, and fixed volumes for soluble proteins and other biochemical components. Current techniques use either spontaneous lipid transfer, peptide-induced fusion, centrifugation or microfluidics to deform lipid monolayers formed at oil-water interfaces and accomplish encapsulation of biomolecules in cell-sized volumes. Techniques based on either small unilamellar vesicles (SUVs, 0.02-0.2 μm in diameter) or giant unilamellar vesicles (GUVs, >10 μm in diameter) can independently achieve asymmetry, encapsulation, and transmembrane protein integration. However, integration of all of these features to construct populations of mono disperse cell-like assemblies has not yet been demonstrated and would be difficult or impossible with existing techniques. Surprisingly, the present invention meets these and other needs.

### BRIEF SUMMARY OF THE INVENTION

In one embodiment, the present invention provides a vesicle having a unilamellar bilayer having a bilayer lipid and at least one bilayer component each independently a membrane protein or a functionalized lipid. The vesicle also includes a component encapsulated by the unilamellar bilayer, wherein the encapsulated component is a protein, a peptide, an enzyme, an oligonucleotide, or a polynucleotide.

In another embodiment, the present invention provides a method of forming a vesicle, the method including contacting an aqueous mixture and an oil mixture, wherein the aqueous mixture includes a first lipid, and the oil mixture includes a second lipid, wherein the aqueous mixture or oil mixture also includes at least one bilayer component of a transmembrane protein or a functionalized lipid. Thus, a lipid bilayer forms at the interface of the aqueous mixture and the oil mixture, wherein the interfacial lipid bilayer includes an aqueous mixture lipid layer having the first lipid, and an oil mixture lipid layer having the second lipid, wherein the interfacial lipid bilayer also includes the membrane protein and the functionalized lipid when present. The method also includes pulsing the interfacial lipid bilayer with a fluid mixture from an inkjet, wherein the fluid mixture includes at least one component of a protein, a peptide, an enzyme, an oligonucleotide, or a polynucleotide. Thus, the vesicle is formed.

### BRIEF DESCRIPTION OF THE DRAWINGS

FIG. 1 shows the formation of lipid vesicles with an inkjet. (a) Each vesicle is formed by a series of inkjet pulses that form a single microfluidic jet, where pauses between pulse sets determine the frequency of vesicle formation. (b) Multiple inkjet pulses (15 pulses at 30 V constant amplitude) build to form a single traveling vortex ring as depicted in sequential frames recorded at 22,500 frames per second (222, 311, 356, 444, 578, 711, 889 μs). (c) A vesicle is formed by a series of 17 inkjet pulses at 50 V, as depicted in sequential frames recorded at 5,000 frames per second (0.8, 1.4, 2.2, 3.0, 4.2, 4.8, 9.4 ms). Scale bars are 100 μm.

FIG. 2 shows the control of vesicle formation through variation of the amplitude and number of inkjet pulses used to form each vesicle. (a) Vortex ring displacement as a function of time for fixed pulse number (15 pulses) and a range of pulse amplitudes. From top to bottom, curves were recorded at: 10 V, 15 V, 20 V, 25 V, and 30 V. (b) Vortex ring displacement as a function of time for fixed pulse amplitude (30 V) and a range of pulse numbers. From top to bottom, curves were recorded with pulse number: 1, 3, 5, 9, 13, and 15. For all ring displacements, the standard deviation between displacement values was less than 2% for N=3. (c) Vesicle diameter as a function of pulse number for fixed pulse amplitude (35 V). Solid line is a power law curve fit with power ⅓, leading coefficient 75.3 μm, and R² of 0.917. (d) Minimum number of pulses to create a vesicle as a function of pulse amplitude in volts. (e) Diameter of vesicle formed using minimum number of pulses, as recorded in part d, as a function of pulse amplitude in volts.

FIG. 3 shows the formation of cell-sized vesicles using a viscosity differential across the bilayer lipid membrane. (a) By placing a solution of elevated viscosity (approximately 3.9 cp) on the side of the bilayer opposite the inkjet, it is possible to manipulate the shear rates on each side of the bilayer during membrane collapse such that small, cell-sized vesicles are formed (8-110 μm diameter). The viscosities of the jetted solution, surrounding solution, and opposite solution are labeled as μj, μi, and μo, respectively. (b) Formation of small cell-sized vesicles as depicted by sequential frames recorded at 7500 frames per second (0.53, 2.00, 2.80, 3.87, 4.53, 5.20, 5.73, 6.27 ms) using inkjet pulses at 36 volts amplitude. (b inset) Enlargement of cell-sized vesicle from a separate formation at higher (20×) magnification. Scale bars are 50 μm.

FIG. 4 shows the formation of multiple vesicles by inkjet printing. (a) Three vesicles are formed at a rate of 200 Hz, as depicted in sequential frames recorded at 5,400 frames per second (1.11, 3.33, 6.67, 9.82, 11.67, 13.70 ms) using 16 inkjet pulses per vesicle at 33 V amplitude. (b) A population of vesicles formed by high throughput inkjet printing. Scale bars are 100 μm.

FIG. 5 shows the controlled assembly and interrogation of actin networks in lipid vesicles. (a) A mixture of actin monomers and 500 nm fluorescent beads are co-encapsulated in vesicles by microfluidic encapsulation with an inkjet. A polymerization buffer is entrained and mixed with the beads and monomers during encapsulation such that network assembly begins only after vesicles are formed. Diffusion of fluorescent beads is a probe for network viscosity. (b) Free diffusion of individual fluorescent beads (marked trails are visible as light grey lines) over 5 s. Scale bar is 10 μm. (c) Diffusion of individual fluorescent beads is confined by actin network polymerization (marked trails are less prominent over the same period). Scale bar is 10 μm. (d) Plot of mean squared bead displacement with (bottom trace) and without (top trace) actin network confinement as a function of time. (e) Zoomed plot of mean squared bead displacement after actin-network assembly. The traces comesfrom three vesicles and represents the average from 30 beads (10 beads within each vesicle).

FIG. 6 shows vortex ring formation and propagation. Individual frames taken from a high-speed movie of jet flow in absence of planar lipid bilayer (9,000 fps; t1-t8 correspond to 222 μs, 333 μs, 444 μs, 667 μs, 889 μs, 1,111 μs, 1,333 μs, and 1,556 μs after the start of actuator expansion). (Scale bar: 100 μm.) Bright-field contrast created by jetting a 200 mM sucrose solution into a surrounding solution of 200 mM glucose.

FIG. 7 shows the failure of vesicle formation upon membrane deformation at an actuator expansion rate 10% less than that required for vesicle formation. Individual frames taken from a high-speed movie (7,500 fps; t1-t8 correspond to 533 μs, 800 μs, 1,067 μs, 2,000 μs, 2,800 μs, 3,867 μs, 4,533 μs, and 5,200 μs after the start of actuator expansion). (Scale bar: 100 μm.) Bright-field contrast was created by jetting a 200 mM sucrose solution into a surrounding solution of 200 mM glucose.

FIG. 8 shows a vesicle formation process in which pearls are formed along the lipid tube. Individual frames taken from a high-speed movie (7,500 fps) of vesicle formation (t1-t8 correspond to 533 μs, 667 μs, 800 μs, 1,467 μs, 1,867 μs, 2,800 μs, 4,667 μs, and 6,933 μs after the start of actuator expansion). (Scale bar: 100 μm.) Bright-field contrast created by jetting a 200 mM sucrose solution in a 200 mM glucose solution.

FIG. 9 shows the formation of water-oil-water emulsions. (a) Frames from high-speed movies (t1-t7 correspond to 800 μs, 1,467 μs, 2,133 μs, 2,667 μs, 2,800 μs, 3,333 μs, and 4,000 μs after the start of actuator expansion). (b) Frames from high-speed movies (t1-t5 correspond to 533 μs,1,067 μs, 1,867 μs, 4,000 μs, and 8,533 μs after the start of actuator expansion). (Scale bars: 100 μm.)

FIG. 10 shows water-oil-water emulsions settled on the chamber bottom after formation. An irregularly shaped, oil-affected contact with the substrate is formed.

FIG. 11 shows GUV formation with oil-insoluble lipids via SUV incorporation into planar bilayers followed by microfluidic jetting. (a) Aqueous droplets containing SUV s with oil-insoluble lipids are incubated in a chamber containing decane oil. A thin acrylic divider separates the two aqueous droplets. SUVs diffuse within the water droplet until they contact and fuse to the oil/water interface, forming a continuous lipid monolayer around each droplet. (b) Removal of the thin acrylic divider allows the two droplets to move together and exclude oil between them. When the two lipid monolayers come into contact, they form a planar lipid bilayer. (c) GUVs are formed by microfluidic jetting of the planar bilayer with an inkjet device that deforms the bilayer into a vesicle that separates from the planar bilayer. Repeated pulsing of the inkjet results in the formation of multiple identical vesicles. (d) Experimental confocal micrscopy data showing incorporation of TMR-PIP2 into a GUV by this method. Scale bar, 50 μm.

FIG. 12 shows formation of GUVs with asymmetric lipid composition via control of the SUV content of each chamber reservoir. (a) SUVs containing Ni-chelating lipids were incubated in the droplet nearest the inkjet (inner droplet). Removal of the divider formed an asymmetric planar bilayer, from which an asymmetric GUV containing Ni-chelating lipids in only its inner leaflet was created by micro fluidic jetting. His-GFP (star icons) was added to the outer droplet after vesicle formation, and the distribution of His-GFP was observed experimentally by confocal microscopy as shown at left. (b) SUVs containing Ni-chelating lipids were incubated in the droplet furthest from the inkjet (outer droplet).

GUVs were again created by microfluidic jetting of the asymmetric planar bilayer, this time containing Ni-chelating lipids in only their outer leaflet. His-GFP (star icons) was added to the outer droplet after vesicle formation, and its distribution was observed experimentally by confocal microscopy as shown at left. All scale bars, 50 μm.

FIG. 13 shows the incorpation of membrane proteins synaptobrevin and syntaxin into GUVs with controlled orientation. (a) Domain structure of GFP-Syb, which was incorporated into GUVs and imaged by confocal microscopy. (b) Domain structure of SybSN-GFP (lacking transmembrane domain), SNAP25 and membrane protein syntaxinΔHabc. When SybSN-GFP was encapsulated into GUVs without tSNAREs, it did not localize to the GUV membrane. However, when SybSN-GFP was encapsulated into GUVs containing SNAP25 and syntaxinΔHabc, it localized to the GUV membrane by forming a SNARE complex with SNAP25 and syntaxinΔHabc. (c) (top row) GUVs made by microfluidic jetting planar bilayers with GFP-Syb SUVs incubated in the outer droplet (left), both droplets (middle), or the inner droplet(right). (bottom row) The same GUVs were imaged again after the addition of Protease K, which degrades exposed protein, to the external medium. (d) GUVs made by incubating GFP-Syb SUVs in the outer droplet had 90 +/−2% (s.e.m., n=6 bilayers) GFP-Syb molecules oriented outwards, with GFP in the extemal lumen. GUVs made by adding GFP-Syb SUVs to both droplets had 51+/−3% (s.e.m., n=4 bilayers) GFP-Syb molecules oriented outwards, and GUVs made by adding GFP-Syb to the inner droplet had 9+/−5% (s.e.m., n=4 bilayers) GFP-Syb molecules oriented outwards. All scale bars, 50 μm. SN: SNARE domain, TM: transmembrane domain.

FIG. 14 shows that Doc2 and SNARE proteins drive docking and fusion of encapsulated SUVs with GUVs. (a) Planar bilayers containing SNAP25 and syntaxinΔHabc (tSNAREs) were preformed by SUV incubation. Doc2 and SUVs incorporating Syb and GFP-Syb (vSNARE SUVs) were loaded into the inkjet and jetted against the planar bilayer to form a GUV containing the proteins. Ca2+ was entrained from the aqueous droplets during the formation process. Domain structures of Syb, GFP-Syb, SNAP25, syntaxinΔHabc, Doc2. (b) GUVs lacking tSNAREs but containing vSNARE SUVs and Doc2 were imaged at time points of 7 minutes and 65 minutes by confocal microscopy. The accumulation of SUVs at the membrane suggests tSNAREs are not required for SUV-GUV docking. GUVs containing tSNAREs and loaded with vSNARE SUVs and Doc2 were also imaged at 10 and 47 minutes. The increase in fluorescence at the membrane confirms that tSNAREs are required for SUV-GUV fusion. (c) Docking of SUVs to a GUV. vSNARE SUVs were co-encapsulated with Doc2 into a GUV and observed by confocal microscopy. Diffusion of an SUV cluster before (dark grey line within GUV) and after (light line along GUV membrane) docking to the GUV membrane was tracked with particle tracking software. The location of the SUV cluster in the first frame is denoted by an ‘x’. Fluorescence intensity of the GUV membrane at the docking location is plotted as a function of time. (d) SNARE mediated fusion of SUVs to a GUV. vSNARE SUVs and Doc2 were loaded into tSNARE GUVs. The path of an SUV cluster was tracked (dark grey line) until fusion with the GUV membrane. The location of the SUV cluster in the first frame is denoted by an ‘x’. Fluorescence intensity of the GUV membrane at the fusion location is plotted as a function of time. All scale bars, 25 μm. SN: SNARE domain, TM: transmembrane domain.

FIG. 15 shows the characterization of GUVs formed by microfluidic jetting. (a) Membrane labeling and volume exclusion is shown for the same GUV. Scale bars are 50 μm. (left) Phase contrast image of the GUV. (center) Labeling of the GUV membrane using BODIPY dye. (right) Wide-field fluorescence image documenting exclusion of sulfo-rhodamine B dye by the GUV. (b) Encapsulation of polystyrene beads into GUVs with the microfluidic jet. (left) Bright-field image of 4 GUVs created from a single planar bilayer. (right) Encapsulation of fluorescent beads (500 nm diameter, FITC) in GUVs.

FIG. 16 shows the protein pore-mediated transport of solutes across vesicle boundaries. (a) Schematic diagram and (b) experimental results showing that a GUV initially excluding FITC dye increases in fluorescence relative to the fluorescence of the external solution after addition of α-hemolysin. α-hemolysin is added to 2.5 μg/ml at time zero and the vesicle is tracked for 104 minutes, at which point relative fluorescence has reached 76%.

FIG. 17 shows the schematic representation of the inkjet vesicle encapsulation system. A bilayer lipid membrane is formed in the acrylic chamber and placed on the stage of an inverted microscope. A conventional disposable syringe is filled with the fluid to be jetted. A fitting is used to attach the syringe to the inkjet device. The syringe plunger is advanced to fill the inkjet device with fluid. The syringe-inkjet assembly is fixed to a syringe support system. Using a 3-axis linear micrometer system (not shown), the tip of the inkjet device is inserted through a hole in the side of the bilayer chamber. To maintain the concentration of the inkjet fluid, a linear motorized actuator is used to advance the syringe plunger, forcing fluid out of the syringe-inkjet assembly. To form a vesicle, an appropriate set of voltage pulses is applied to the piezoelectric tube of the inkjet device. This voltage pulse causes expansions and contractions of the piezoelectric tube, which propel a fluid jet that impinges upon the bilayer lipid membrane, forming a vesicle.

### DETAILED DESCRIPTION OF THE INVENTION

I. General

The present invention describes vesicles having complex functionality allowing the vesicles to mimic properties and activities of biological cells. The vesicles of the present invention are artificial cells. For example, the vesicles can incorporate multiple biological components in the vesicle interior, and proteins, such as membrane proteins, and/or functionalized lipids in the vesicle bilayer. The functionalized lipids of the vesicle bilayer can include signalling or chelating moieties that provide the additional functionality for the vesicles of the present invention. Moreover, the vesicle bilayer can be modified asymmetrically by selective addition of a protein or functionalized lipid to either the aqueous or oil-mixture during vesicle preparation. In particular, the preparation methods of the present invention enable the incorporation of oil-insoluble lipids, such as anionic lipids, into the vesicle bilayer. The orientation of membrane proteins in the vesicle bilayer can be similarly controlled.

II. Definitions

“Lipid” refers to a small molecule having hydrophobic or amphiphilic properties and is useful for preparation of vesicles, micelles and liposomes. Lipids include, but are not limited to, fats, waxes, fatty acids, cholesterol, sphingolipids, phospholipids, monoglycerides, diglycerides and triglycerides. Phospholipids, sphingolipids, and glycerides contain fatty acid chains that can be saturated, mono-unsaturated, or poly-unsaturated. Branched-chain fatty acids, including but not limited to isoprenoid fatty acids and mycolic acids, may contain pendant groups, including hydroxy groups and alkyl groups, substituting the hydrocarbon chains.

Lipids can be “uncharged lipids” or “charged lipids.” “Uncharged lipids” refer to lipids that do not carry any charged or ionizable groups such as phosphate groups or choline groups. Examples of uncharged lipids include, but are not limited to, diacyl glycerols and prostaglandins. “Charged lipids” include zwitterionic lipids, cationic lipids and anionic lipids. Zwitterionic lipids carry both positively-charged groups and ionizable groups such as amino groups and choline groups that bear a net positive charge, and negatively-charged groups and ionizable groups, such as phosphates, sulfates and carboxylates. Examples of zwitterionic lipids include, but are not limited to, phosphorylcholine and phosphorylethanolamine Cationic lipids carry positively-charged groups and ionizable groups and bear a net positive charge. Examples of cationic lipids include, but are not limited to, dimethyldioctadecylammonium bromide and ethyl phosphatidyl dicholine. Anionic lipids carry negatively-charged groups and ionizable groups such as phosphate groups and bear a net negative charge. Examples of anionic lipids include, but are not limited to, phosphatidylinositol 4,5-bisphosphate (PIP₂) and phosphatidylglycerol.

“Functionalized lipids” includes lipids that are modified with natural, or physiological groups, or non-natural groups. In the case of amphiphilic lipids such as phospholipids and sphingolipids, modification can be made to either the polar head group or the nonpolar fatty acid chains. Modified lipids include, but are not limited to, polymer-modified lipids such as 1,2-dimyristoyl-sn-glycero-3-phosphoethanolamine-N-[methoxy(polyethylene glycol)-2000], chelating lipids such as (1,2-dioleoyl-sn-glycero-3-[(N-(5-amino-1-carboxypentyl)-iminodiacetic acid)succinyl])-Ni, and fluorescent lipids such as tetramethylrhodamine-phosphatidylinositol(4,5)-bisphosphate and 2-dioleoyl-sn-glycero-3-phosphoethanolamine-N-(7-nitro-2-1,3-benzoxadiazol-4-yl). Functionalized lipids also include those that are modified with physiological groups to form a signaling lipid.

“Vesicle”refers to a non-natural or synthetic membranous and usually fluid-filled pouch resulting from the supramolecular assembly of lipids including, but not limited to, phospholipids. The interior contents of a phospholipid vesicle are separated from the exterior surroundings by at least one phospholipid bilayer. A phospholipid bilayer is a sheet of lipids two molecules thick, arranged so that the hydrophilic phosphate heads point “out” to the solution on either side of the bilayer and the hydrophobic tails point “in” to the core of the bilayer. This results in two “leaflets” which are each a single molecular layer of phospholipids. A “unilamellar vesicle” refers to a vesicle comprising only one phospholipid bilayer; “multilamellar vesicle” refers to a vesicle comprising more than one phospholipid bilayer. A “symmetric bilayer” is defined as a bilayer posessing two leaflets of the same composition, whereas an “asymmetric bilayer” is defined as a bilayer possessing two leaflets which differ in composition.

“Protein” and “peptide” are used interchangeably herein to refer to a polymer of amino acid residues. These terms apply to naturally occurring amino acid polymers and non-naturally occurring amino acid polymers, as well as to amino acid polymers in which one or more amino acid residue is an artificial chemical mimetic of a corresponding naturally occurring amino acid. As used herein, the terms encompass amino acid chains of any length, including full-length proteins, wherein the amino acid residues are linked by covalent peptide bonds.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

“Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

“Unnatural amino acids” are not encoded by the genetic code and can, but do not necessarily have the same basic structure as a naturally occurring amino acid. Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, ornithine, pentylglycine, pipecolic acid and thioproline.

“Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid (i.e., hydrophobic, hydrophilic, positively charged, neutral, negatively charged). Exemplified hydrophobic amino acids include valine, leucine, isoleucine, methionine, phenylalanine, and tryptophan. Exemplified aromatic amino acids include phenylalanine, tyrosine and tryptophan. Exemplified aliphatic amino acids include serine and threonine. Exemplified basic amino acids include lysine, arginine and histidine. Exemplified amino acids with carboxylate side-chains include aspartate and glutamate. Exemplified amino acids with carboxamide side chains include asparagines and glutamine Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

“Enzyme” refers to a protein that catalyzes a chemical reaction.

“Membrane protein” refers to a protein molecule that is attached to, or associated with, the membrane of a cell, organelle, or other vesicle. Membrane proteins include “integral membrane proteins” which penetrate the lipid bilayer. “Integral polytopic proteins” (also called transmembrane proteins) span both leaflets of a lipid bilayer, while “integral monotopic proteins” are attached to a bilayer from a single leaflet and do not span the entire membrane. Examples of transmembrane proteins include, but are not limited to, syntaxinla and synaptobrevin (Syb). Membrane proteins also include “lipid-anchored proteins,” which are proteins with one or more covalently attached fatty acid molecules that anchor to either leaflet of a lipid membrane. One example of a lipid-anchored protein is SNAP25, which is tethered to lipid membranes via several cysteine-linked palmitoyl chains.

Certain membrane proteins are defined as “SNAP Receptor proteins” and are referred to herein as “SNARE proteins.” SNARE proteins make up a large protein superfamily whose primary role is to mediate the fusion of cellular transport vesicles with target membranes such as the cell membrane during exocytosis, or with the membranes of other compartments such as lysosomes. SNARE proteins are divided into two categories known as “tSNAREs” and “vSNAREs.” vSNARES include proteins bound to vesicle membranes and include, but are not limited to, Syb. tSNARES include proteins bound to membranes of target compartments and include, but are not limited to syntaxinla and SNAP25.

The terms “nucleic acid,” “oligonucelotide,” and “polynucleotide” refer to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

“Diagnostic agent” refers to an agent capable of diagnosing a condition or disease. Diagnostic agents include, but are not limited to, chromophores, fluorophores, and radiolabels.

“Therapeutic agent” refers to an agent capable of treating and/or ameliorating a condition or disease. Therapeutic agents include, but are not limited to, compounds, drugs, peptides, oligonucleotides, DNA, antibodies, and others.

“Fluid mixture” refers to an oil- or water-based solvent used to form the vesicle interior of the present invention. The fluid mixture can include a variety of components including, but not limited to, proteins, or nucleic acids. Preferred fluid mixtures contain proteins in aqueous solution and are used for formation of giant unilamellar vesicles.

“Aqueous mixture” refers to a solution or suspension of lipids or other molecules substantially in water. The aqueous mixtures are immiscible with the oil-based mixtures of the present invention.

“Oil mixture” refers to a solution or suspension of lipids or other molecules in water-immiscible solvents. Exemplary water-immiscble solvents, referred to as “oils,” suitable for the preparation of certain lipid membranes and vesicles in the present invention include, but are not limited to, chloroform and hydrocarbons such as decane. One of skill in the art will appreciate that other solvents are useful as the oil mixture in the present invention.

“Contacting” refers to the process of bringing into contact at least two immiscible mixtures each containing a lipid, such that a lipid bilayer is formed at the interface of the two mixtures.

“Interface” is defined as the area of contact between two or more entities that possess distinct boundaries. In certain cases, the interface is a small contact area between closely opposed compartments resulting from the exclusion of the surrounding medium.

“Interfacial lipid bilayer” refers to a lipid bilayer that forms at the point of contact between closely opposed liquid compartments in a surrounding medium. In preferred embodiments of the invention, planar lipid bilayers are created at the interface between two aqueous droplets that are initially surrounded by oil containing dissolved lipids and then brought into contact to exclude the surrounding oil.

“Inkjet” refers to a fluid-filled chamber containing a piezoelectric element and connected to a nozzle. When a voltage is applied the piezoelectric response generates a pressure pulse in the fluid, forcing it from the chamber through the nozzle orifice. As used herein, the term may also refer to the process of employing an inkjet for the formation of giant unilamellar vesicles.

“Pulsing” refers to the application of voltage pulses to a cylindrical piezoelectric actuator surrounding a fluid-filled nozzle in an injket. The actuator contracts and expands radially, producing pressurization and rarefaction waves in the fluid. Application of appropriate voltage pulses to such devices results in the ejection of fluid from the device due to the constructive interference of traveling pressurization waves (and destructive interference of traveling rarefaction waves) within the nozzle. The fluid pulses travel in and entrain the surrounding medium, combining to form a vortex ring structure that is capable of deforming bilayer lipid membranes to form vesicles.

III. Vesicle Composition

The vesicle compositions of the present invention include giant unilamellar vesicles (GUVs) having complex functionality that mimics biological cells. In some embodiments, the vesicles of the present invention are artificial cells. The vesicle of the present invention can have a unilamellar bilayer including a lipid and a component encapsulated by the bilayer. The encapsulated component can be a protein, a peptide, an enzyme, an oligonucleotide, or a polynucleotide.

In some embodiments, the present invention provides a vesicle having a unilamellar bilayer having a bilayer lipid and at least one bilayer component each independently a membrane protein or a functionalized lipid. The vesicle also includes a component encapsulated by the unilamellar bilayer, wherein the encapsulated component is a protein, a peptide, an enzyme, an oligonucleotide, or a polynucleotide.

### Lipids

The lipids of the present invention are small molecules having hydrophobic or amphiphilic properties. Lipids include, but are not limited to, fats, waxes, fatty acids, cholesterol, sphingolipids, phospholipids, monoglycerides, diglycerides and triglycerides. Phospholipids, sphingolipids, and glycerides contain fatty acid chains that can be saturated, mono-unsaturated, or poly-unsaturated. Branched-chain fatty acids, including but not limited to isoprenoid fatty acids and mycolic acids, can contain pendant groups, including hydroxy groups and alkyl groups, substituting the hydrocarbon chains. Lipids useful in the present invention also include partially unsaturated alkyl chains. Examples of fatty acids include but are not limited to capric acid (C10), lauric acid (C12), myristic acid (C14), palmitic acid (C16), palmitoleic acid (C16), stearic acid (C18), isostearic acid (C18), oleic acid (C18), vaccenic acid (C18), linoleic acid (C18), alpha-linoleic acid (C18), gamma-linolenic acid (C18), phytanic acid (C20) arachidic acid (C20), gadoleic acid (C20), arachidonic acid (C20), eicosapentaenoic acid (C20), behenic acid (C22), erucic acid (C22), docosahexaenoic acid (C22), lignoceric acid (C24) and hexacosanoic acid (C26). In some embodiments, the bilayer lipid can be 1,2-diphytanoyl-sn-glycero-3-phosphocholine (DPhPC).

In certain embodiments, lipids can include those typically present in cellular membranes, such as phospholipids and/or sphingolipids. Suitable phospholipids include but are not limited to phosphatidylcholine (PC), phosphatidic acid (PA), phosphatidylethanolamine (PE), phosphatidylglycerol (PG), phosphatidylserine (PS), and phosphatidylinositol (PI). Suitable sphingolipids include but are not limited to sphingosine, ceramide, sphingomyelin, cerebrosides, sulfatides, gangliosides, and phytosphingosine. Other suitable lipids can include lipid extracts, such as egg PC, heart extract, brain extract, liver extract, and soy PC. In some embodiments, soy PC can include Hydro Soy PC (HSPC).

In certain embodiments, the lipid composition of a vesicle of the present invention can be tailored to affect characteristics of the vesicles, such as leakage rates, stability, particle size, zeta potential, protein binding, in vivo circulation, and/or accumulation in tissue, such as a tumor, liver, spleen or the like. For example, DSPC and/or cholesterol can be used to decrease leakage. Negatively or positively lipids, such as DSPG and/or DOTAP, can be included to affect the surface charge. In some embodiments, the vesicles can include about ten or fewer types of lipids, or about five or fewer types of lipids, or about three or fewer types of lipids. In some embodiments, the molar percentage (mol %) of a specific type of lipid present typically comprises from about 0% to about 10%, from about 10% to about 30%, from about 30% to about 50%, from about 50% to about 70%, from about 70% to about 90%, from about 90% to 100% of the total lipid present in vesicle.

Lipids can be uncharged or charged. Uncharged lipids carry no charged or ionizable groups such as phosphate groups or choline groups. Examples of this type of lipid include, but are not limited to, diacyl glycerols (such as 1-palmitoyl-2-oleoyl-sn-glycerol) and prostaglandins (such as PGE1, PGF1α, and PGF1β). Other lipids useful in the present invention include, but are not limited to, dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin. In certain embodiments, the lipids can include derivatized lipids, such as PEGlyated lipids. Derivatized lipids can include, for example, DSPE-PEG2000, cholesterol-PEG2000, DSPE-polyglycerol, or other derivatives generally well known in the art.

Charged lipids can be neutrally charged (zwitterionic), or have a net anionic or cationic charge. Zwitterionic lipids carry charged or ionizable groups but have a net neutral charge under appropriate environmental conditions. These are termed zwitterionic lipids. Examples of zwitterionic lipids include phosphatidylcholines including, but not limited to, POPC (1-palmitoyl-2-oleoyl-sn-glycero-3-phosphocholine), DMPC (1,2-dimyristoyl-sn-glycero-3-phosphocholine), DPPC (1,2-dipalmitoyl-sn-glycero-3-phosphocholine), and DOPC (1,2-dioleoyl-sn-glycero-3-phosphocholine). Zwitterionic lipids also include lysophosphatidylcholines such as 1-palmitoyl-sn-glycero-3-phosphocholine, phosphatidylethanolamines including, but not limited to, SOPE (1-stearoyl-2-oleoyl-sn-glycero-3-phosphoethanolamine), DMPE (1,2-dimyristoyl-sn-glycero-3-phosphoethanolamine), DPPE (1,2-dipalmitoyl-sn-glycero-3-phosphoethanolamine), and DOPE (1,2-dioleoyl-sn-glycero-3-phosphoethanolamine), and lysoethanolamines including 1-stearoyl-sn-glycero-3-phosphoethanolamine In some embodiments, the lipid can be DPhPC (1,2-diphytanoyl-sn-glycero-3-phosphocholine).

Other charged lipids include cationic lipids and anionic lipids. Cationic lipids carry positively-charged groups and ionizable groups such as amino groups and choline groups and bear a net positive charge. Examples of cationic lipids include, but are not limited to, DDAB (dimethyldioctadecylammonium bromide), DOSPA (2,3-dioleyloxy-N-[2(spermine-carboxamido)ethyl]-N,N-dimethyl-1-propanaminium trifluoroacetate), and O-ethylphosphatidylcholines such as 1-palmitoyl-2-oleoyl-sn-glycero-3-ethylphosphocholine. Other cationic lipids include but are not limited to N,N-dioleoyl-N,N-dimethylammonium chloride (DODAC), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), and N,N-dimethyl-2,3-dioleyloxy)propylamine (DODMA).

Anionic lipids carry negatively-charged groups and ionizable groups such as phosphate groups and bear a net negative charge. Examples of anionic lipids include, but are not limited to, phosphatidic acids such as POPA (1-palmitoyl-2-oleoyl-sn-glycero-3-phosphate) and lysophosphatidic acids such as 1-oleoyl-2-hydroxy-sn-glycero-3-phosphate; phosphatidylglycerols such as POPG (1-palmitoyl-2-oleoyl-sn-glycero-3-phospho-(1′-rac-glycerol) and lysophosphatidylglycerols such as 1-palmitoyl-2-hydroxy-sn-glycero-3-phospho-(1′-rac-glycerol); phosphatidylserines such as SOPS (1-stearoyl-2-oleoyl-sn-glycero-3-phospho-L-serine) and lysophosphatidylserines such as 1-stearoyl-sn-glycero-3-phospho-L-serine; phosphatidylinositols such as 1-stearoyl-2-arachidonoyl-sn-glycero-3-phospho-(1′-myo-inositol); phosphatidylinositolphosphates such as 1-stearoyl-2-arachidonoyl-sn-glycero-3-phospho-(1′-myo-inositol-4′phosphate), 1-stearoyl-2-arachidonoyl-sn-glycero-3-phospho-(1′-myo-inositol-4′,5′-bisphosphate) and phosphatidylinositol 4,5-bisphosphate (PIP₂); and cardiolipins such as 1′,3′-bis[1,2-dilinoleoyl-sn-glycero-3-phospho]-sn-glycerol. Generally, lipids bearing a net positive or negative charge exhibit poor solubility in oil phases.

In certain embodiments of the inventions, lipids can be functionalized with natural or physiological group. In other embodiments, the lipids can be functionalized with non-natural groups such as polymers and chelating groups. In some embodiments, the functionalized lipid can be a PEGylated lipid, a signaling lipid, or a chelating lipid. In other embodiments, the functionalized lipid can be a PEGylated lipid, tetramethylrhodamine-phosphatidylinositol(4,5)-bisphosphate (TMR-PIP₂), or (1,2-dioleoyl-sn-glycero-3-[(N-(5-amino-1-carboxypentyl)-iminodiacetic acid)succinyl]) (DOGS-NTA).

In the case of amphiphilic lipids such as phospholipids and sphingolipids, modification can be made to either the polar head group or the nonpolar fatty acid chains. Functionalized lipids can include glycolsylated lipids such as N-lactosylphosphatidylethanolamines, and lipids functionalized with polyethylene glycol (PEG). PEGylated lipids include, but are not limited to DPPC-PEG2k. Functionalized lipids can also include polymer-modified lipids such as (1,2-distearoyl-sn-glycero-3-phosphoethanolamine-N-[PEG2000-N′-carboxyfluorescein]. Preferred functionalized lipids can include a chelating lipid such as DOGS-NTA-Ni ((1,2-dioleoyl-sn-glycero-3-[(N-(5-amino-1-carboxypentyl)-iminodiacetic acid)succinyl])-Ni), or a fluorescent lipid such as TMR-PIP₂ (BODIPY-tetramethylrhodamine-phosphatidylinositol-(4,5)-bisphosphate) or DPPE-NBD (2-dioleoyl-sn-glycero-3-phosphoethanolamine-N-(7-nitro-2-1,3-benzoxadiazol-4-yl)). Other functionalized lipids are useful in the compositions and methods of the present invention.

In certain embodiments, the present invention provides membranes and vesicles having at least one phospholipid bilayer. A phospholipid bilayer is a sheet of lipids two molecules thick, arranged so that the hydrophilic phosphate heads point out to the solution on either side of the bilayer and the hydrophobic tails point in to the core of the bilayer. This results in two leaflets which are each a single molecular layer. Preferred bilayer membranes and vesicles contain the phospholipids and functionalized phospholipids described above, and can also include other components such as cholesterol or a membrane protein.

In other embodiments, the bilayer also includes the functionalized lipid selected from a PEGylated lipid, a signaling lipid and a chelating lipid. In other embodiments, the functionalized lipid can be a PEGylated lipid, tetramethylrhodamine-phosphatidylinositol(4,5)-bisphosphate (TMR-PIP₂), or (1,2-dioleoyl-sn-glycero-3-[(N-(5-amino-1-carboxypentyl)-iminodiacetic acid)succinyl])-Ni (DOGS-NTA-Ni)

### Membrane Proteins

In some embodiments, the bilayer component is a membrane protein. The membrane proteins can be attached to, or associated with, the membrane of a cell, organelle, or other vesicle. Membrane proteins include integral membrane proteins which penetrate the lipid bilayer. Integral membrane proteins can attach to a bilayer via a single leaflet without spanning the entire membrane, or can span both leaflets of the bilayer, in which case they are referred to as transmembrane proteins. Examples of transmembrane proteins include, but are not limited to, syntaxinla and synaptobrevin (Syb). Certain transmembrane proteins, such as α-hemolysin (a bacterial endotoxin) and various ion channel proteins, can form pores in the phospholipid bilayer. Membrane proteins also include lipid-anchored proteins, which include proteins with one or more covalently attached fatty acid molecules that anchor to either leaflet of a lipid membrane. Examples of lipid-anchored proteins include HRas, which help regulate cell division, and certain G proteins which participate in cell-signalling pathways. A preferred lipid-anchored protein in the present invention is SNAP25, which is tethered to lipid membranes via several cysteine-linked palmitoyl chains.

In some embodiments, the membrane protein can be soluble N-ethylmaleimide-sensitive factor attachment protein receptor (SNARE) protein synaptobrevin. The primary role of SNARE proteins is to mediate the fusion of cellular transport vesicles with target membranes such as the cell membrane during exocytosis, or with the membranes of other compartments such as lysosomes. vSNARES are SNARE proteins that are bound to vesicle membranes and include, but are not limited to, Syb. tSNARES are SNARE proteins that are bound to membranes of target compartments and include, but are not limited to, syntaxinl a and SNAP25.

### Bilayer Symmetry

The vesicles of the present invention include at least one lipid bilayer. In some embodiments, the lipid bilayer is unilamellar, having only a single lipid bilayer. In other embodiments, the lipid bilayer is multilamellar, having more than one lipid bilayer. The lipid bilayer of the present invention can be symmetric or asymmetric, depending on the composition of the inner and outer leaflets of the lipid bilayer. A symmetric bilayer includes two leaflets of the same composition, while an asymmetric bilayer includes two leaflets having different compositions. Asymmetric bilayers can be formed so as to selectively functionalize the inner leaflet or the outer leaflet of the phospholipid membranes. In some embodiments, the vesicles include asymmetric unilamellar lipid bilayers.

The vesicles of the present invention include an interior and an exterior, such that when the lipid bilayer is asymmetric and includes a membrane protein, more than 50% of the membrane protein can be oriented towards either the interior or exterior of the vesicle. In other embodiments, more than 60, 70, 80, 90 or 95% of the membrane protein can be oriented towards the interior or exterior of the vesicle.

As described above, the vesicle bilayer includes two leaflets, the inner leaflet closest to the vesicle interior, and the outer leaflet closest to the vesicle exterior. Depending on the composition of each leaflet, the vesicle can be symmetric, when the leaflets have the same composition, or asymmetric, when the leaflets have different compositions. In some embodiments, the unilamellar bilayer also includes an inner leaflet and an outer leaflet, wherein the outer leaflet includes the bilayer lipid and the functionalized lipid; and the inner leaflet includes the bilayer lipid, thereby forming an asymmetric unilamellar bilayer. In other embodiments, the unilamellar bilayer also includes an inner leaflet and an outer leaflet, wherein the outer leaflet includes the bilayer lipid; and the inner leaflet includes the bilayer lipid and the functionalized lipid, thereby forming an asymmetric unilamellar bilayer. The leaflet composition can include additional components, or can be substantially limited to the components described above. The inner and outer leaflets can include other components. In some embodiments, the inner and outer leaflets are limited to the components described above. For example, in some embodments, the outer leaflet consists essentially of the bilayer lipid and the functionalized lipid; and the inner leaflet consists essentially of the bilayer lipid, thereby forming an asymmetric unilamellar bilayer. In other embodiments, the unilamellar bilayer also includes an inner leaflet and an outer leaflet, wherein the outer leaflet consists essentially of the bilayer lipid; and the inner leaflet consists essentially of the bilayer lipid and the functionalized lipid, thereby forming an asymmetric unilamellar bilayer.

### Encapsulated Components

In some embodiments, the vesicles of the present invention are particles whose interior contents are separated from the exterior surroundings by at least one phospholipid bilayer.

The vesicles of the present invention also contain encapsulated components which include, but are not limited to, proteins, peptides, enzymes, oligonucleotides, polynucleotides, diagnostic agents, and therapeutic agents. In some embodiments, the vesicles include two or more encapsulated components. In other embodiments, the unilamellar bilayer encapsulates a second component such as a protein, a peptide, an enzyme, an oligonucleotide, and a polynucleotide.

Peptides and proteins can be synthesized chemically or expressed and purified from recombinant and/or native organisms. Preferred proteins encapsulated in the vesicles are green fluorescent protein (GFP) and related fusion proteins, as well as the SNARE-related protein Doc2. Encapsulated proteins can also be structural proteins including, but not limited to, actin, tubulin, lamins, and keratins. A preferred structural protein in the present invention is actin. Encapsulated proteins can be enzymes which catalyze chemical reactions. Encapsulated enzymes can include, but are not limited to, proteases, glycosidases, and restriction enzymes and other nucleases. Proteins can also be encapsulated in vesicles in the form of proteo-liposomes. In preferred embodiments of the present invention, proteo-liposomes contain vSNARE proteins such as Syb and related fusion proteins.

Encapsulated oligonucelotides and polynucleotides of the present invention are polymers of deoxyribonucleic acids (DNA) or ribonucleic acids (RNA) in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Nucleic acids sequences may be naturally occuring and/or engineered using recombinant technologies known in the art. Oligonucleotides and polynucleotides may be chemically synthesized or isolated from tissues, cell cultures, or other sources. Examples of encapsulated nucleic acids include, but are not limited to, plasmid DNA, siRNA, ribozymes, and aptamers.

The vesicles of the present invention can also encapsulate other components known to one of skill in the art, such as salts, buffers, and other pharmaceutically acceptable excipients. Exemplary salts include Ca²⁺.

### Therapeutic & Diagnostic Agents

In some embodiments, the vesicles of the present invention can also include a diagnostic agent or a therapeutic agent. Diagnostic agents can be chromophores and fluorophores which include, but are not limited to, xanthene, cyanine, and oxazine derivatives. Diagnostic agents can also be MRI contrast reagents such as gadopentetic acid and radiotracers such as fluorodeoxyglucose (¹⁸F). Therapeutic agent are capable of treating and/or ameliorating a condition or disease. Therapeutic agents include, but are not limited to, compounds, drugs, peptides, oligonucleotides, DNA, antibodies, and others.

The vesicles of the present invention can include a therapeutic agent, diagnostic agent, or a combination thereof A therapeutic agent used in the present invention can include any agent directed to treat a condition in a subject. In general, any therapeutic agent known in the art can be used, including without limitation agents listed in the United States Pharmacopeia (U.S.P.), Goodman and Gilman's The Pharmacological Basis of Therapeutics, 10^{th} Ed., McGraw Hill, 2001; Katzung, Ed., Basic and Clinical Pharmacology, McGraw-Hill/Appleton & Lange, 8^{th} ed., Sep. 21, 2000; Physician's Desk Reference (Thomson Publishing; and/or The Merck Manual of Diagnosis and Therapy, 18^{th} ed., 2006, Beers and Berkow, Eds., Merck Publishing Group; or, in the case of animals, The Merck Veterinary Manual, 9^{th} ed., Kahn Ed., Merck Publishing Group, 2005; all of which are incorporated herein by reference.

Therapeutic agents can be selected depending on the type of disease desired to be treated. For example, certain types of cancers or tumors, such as carcinoma, sarcoma, leukemia, lymphoma, myeloma, and central nervous system cancers as well as solid tumors and mixed tumors, can involve administration of the same or possibly different therapeutic agents. In certain embodiments, a therapeutic agent can be delivered to treat or affect a cancerous condition in a subject and can include chemotherapeutic agents, such as alkylating agents, antimetabolites, anthracyclines, alkaloids, topoisomerase inhibitors, and other anticancer agents. In some embodiments, the agents can include antisense agents, microRNA, siRNA and/or shRNA agents.

In some embodiments, a therapeutic agent can include an anticancer agent or cytotoxic agent. Therapeutic agents of the present invention can also include radionuclides for use in therapeutic applications. For example, emitters of Auger electrons, such as ¹¹In, can be combined with a chelate, such as diethylenetriaminepentaacetic acid (DTPA) or 1,4,7,10-tetraazacyclododecane-1,4,7,10-tetraacetic acid (DOTA), and included in a targeted delivery composition, such as a liposome, to be used for treatment. Other suitable radionuclide and/or radionuclide-chelate combinations can include but are not limited to beta radionuclides (¹⁷⁷Lu, ¹⁵³Sm, ^{88/90}Y) with DOTA, ⁶⁴Cu-TETA, ^{188/186}Re(CO)₃-IDA; ^{188/186}Re(CO)triamines (cyclic or linear), ^{188/186}Re(CO)₃-Enpy2, and ^{188/186}Re(CO)₃-DTPA.

A diagnostic agent used in the present invention can include any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like.

In some embodiments, a diagnostic agent can include chelators that bind, e.g., to metal ions to be used for a variety of diagnostic imaging techniques. Exemplary chelators include but are not limited to ethylenediaminetetraacetic acid (EDTA), [4-(1,4,8,11-tetraazacyclotetradec-1-yl) methyl]benzoic acid (CPTA), Cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7, 10-tetraazacyclododecane-1,4,7,10-tetra(methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

A radioisotope can be incorporated into some of the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹² Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. In some embodiments, the vesicles can be radiolabeled, for example, by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof.

In yet other embodiments, the diagnostic agents can include but are not limited to magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5^{th} Ed., Blackwell Publishing (2004)). In some embodiments, a diagnostic agent can include a magnetic resonance (MR) imaging agent. Exemplary magnetic resonance agents include but are not limited to paramagnetic agents, superparamagnetic agents, and the like. Exemplary paramagnetic agents can include but are not limited to Gadopentetic acid, Gadoteric acid, Gadodiamide, Gadolinium, Gadoteridol, Mangafodipir, Gadoversetamide, Ferric ammonium citrate, Gadobenic acid, Gadobutrol, or Gadoxetic acid. Superparamagnetic agents can include but are not limited to superparamagnetic iron oxide and Ferristene. In certain embodiments, the diagnostic agents can include x-ray contrast agents as provided, for example, in the following references: H. S Thomsen, R. N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexol, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexol, iopentol, ioversol, iobitridol, iodixanol, iotrolan and iosimenol.

In some embodiments, the unilamellar bilayer also includes an inner leaflet and an outer leaflet, wherein the unilamellar bilayer includes diphytanoylphosphatidylcholine (DPhPC) and tetramethylrhodamine-phosphatidylinositol(4,5)-bisphosphate (TMR-PIP₂). In other embodiments, the unilamellar bilayer includes DPhPC and (1,2-dioleoyl-sn-glycero-3-[(N-(5-amino-1-carboxypentyl)-iminodiacetic acid)succinyl])-Ni (DOGS-NTA-Ni), such that the outer leaflet includes DPhPC and the inner leaflet includes DPhPC and DOGS-NTA-Ni, and optionally encapsulating His-green fluorescent protein. In some other embodiments, the unilamellar bilayer includes DPhPC and DOGS-NTA-Ni, such that the outer leaflet includes DPhPC and DOGS-NTA-Ni, and the inner leaflet includes DPhPC, encapsulating iodixanol and optionally His-green fluorescent protein. In still other embodiments, the unilamellar bilayer includes DPhPC, diphytanoylphosphatidylserine (DPhPS), cholesterol, and with the membrane protein trans-soluble N-ethylmaleimide-sensitive factor attachment protein receptor (tSNARE), encapsulating SybSN-GFP and iodixanol. In yet other embodiments, the unilamellar bilayer includes DPhPC, DPhPS, cholesterol, the membrane protein tSNARE, encapsulating iodixanol, and functionalized on the outer leaflet with SybSN-GFP.

### Vesicle Size

The vesicles of the present invention can be any suitable size. For example, the vesicles can be from about 0.1 μm to about 5 mm in diameter. In some embodiments, the vesicles can be from about 0.1 μm to about 500 μm in diameter. In other embodiments, the vesicles can be from about 1 μm to about 100 μm in diameter. In some other embodiments, the vesicles can be from about 1 μm to about 50 μm in diameter, emulating eukaryotic and some bacterial cells. In still other embodiments, the vesicles of the present invention can be from about 10 nm to about 100 nm in diameter.

IV. Uses of GUVs

The present invention provides vesicles that find use in the fields of engineering, physical and life sciences, and medicine. Certain embodiments provide a means for the reconstitution and study of fundamental biological processes such as such as exocytosis, antigen presentation, viral entry, and signal transduction. Vesicles encapsulating biologically-active compounds can also be used as chemical micro-reactors. Medical applications of lipid vesicals can include drug delivery and diagnostic imaging. The opportunity to combine multiple capabilities to address specific clinical needs makes liposomes attractive therapeutic and diagnostic vehicles. The lipid bilayer membrane boundary of liposomes keeps their contents concentrated and shielded from exposure as they pass through the body. Additionally, modifications of the membrane can be made to improve drug bioavailability and reduce side effects in some clinical applications. When encapsulated in polyethyleneglycol-coated liposomes, the anticancer drug Doxorubicin, has been shown to achieve much better performance in tumor targeting and reduced cardio toxicity compared to the unencapsulated form. Additional modification of the vesicle's bilayer with antibodies, aptamers, or ligands allows encapsulated drugs to be targeted for delivery to sites within the body. Further functionality can be achieved by adding probes that facilitate medical imaging. Liposomes labeled with radio nuclides can be imaged using single-photon emission computed tomography and magnetic resonance imaging, and radiolabeled liposomes containing anti-cancer therapeutics offer the ability to actively evaluate liposomal drug delivery.

V. Methods of Making Vesicles

The vesicles of the present invention can be prepared by a variety of methods. For example, the vesicles can be prepared using techniques such as a liquid jet generated by a piezoelectrically actuated plunger-syringe system to deform a planar lipid bilayer to make unilamellar vesicles loaded with contents of unrestricted size (Stachowiak et al., 2008 and 2009). The method enables the formation of unilamellar vesicles with a high encapsulation efficiency and homogeneous size distribution at a high throughput rate (up to 200 Hz). Vesicles on the scale of micrometers to tens of micrometers can be used directly for cell-like reconstitution, in which the spatial organization and reaction dynamics of cellular structures such as those formed by the cytoskeleton can be studied. To obtain vesicles on the scale of 100 nm (liposomes), the vesicles can be sonicated or extruded as noted earlier. Certain embodiments of the present invention provide cell-sized vesicles through variation of fluid viscosity without the need for sonication or extrusion.

The method of the present invention is based on the ability to precisely form and control a liquid jet using an inkjet drop-on-demand device. This inkjet device consists of a glass capillary surrounded by a cylindrical piezoelectric sleeve. Excitation of the piezoelectric actuator, by application of a voltage pulse, forces the piezoelectric sleeve to contract and expand around the glass capillary causing acoustic compression and rarefaction waves to propagate laterally along the nozzle. In the present invention, the inkjet is submerged in a miscible fluid such that there is no surface tension at the orifice. Therefore, fluid ejection is achieved by a smaller compression amplitude than typical inkjet-based techniques and requires less optimization of rarefraction wave interference.

The voltage profile used to excite the piezoelectric actuator is designed through software controls. Voltage profiles can include a positive amplitude, or upright, trapezoidal profile followed by a negative amplitude, or inverted, trapezoidal profile. A preferred voltage profile employs an upright trapezoidal shape without an inverted profile. Adequate ejection of fluid to form a vesicle generally requires the application of numerous pulses of the trapezoidal waveform (Stachowiak et al., 2009). Preferred profile parameters include a 3 ms rise time from 0 to 35 V, a dwell time of 30 μs, and a fall time of 3 μs 35 to 0 V, repeated for 20 pulses at a frequency of 20 kHz. Parameters can be varied to optimize vesicle formation depending on the distance between the inkjet tip and bilayer, the viscosity of the solutions, and composition the bilayer.

In addition to the high-speed jet formed by the piezoelectric actuator, a continuous slow nozzle flow is used to prevent diffusion of outside fluid into the nozzle of the inkjet. Positive pressure inside the nozzle minimizes clogs in the nozzle due to particles in the solution surrounding the inkjet. If the solution inside the inkjet is different than the surrounding solution, the flow can be used to manipulate the solution concentration around the tip orifice. To enable continuous nozzle flow, the rear of the inkjet is attached by a Luer-Lock fitting to a syringe. A motorized linear actuator connected to the plunger of the syringe allows the user to drive fluid from the syringe through the inkjet and out the front of the nozzle prior to inkjet actuation and vesicle formation. In preferred embodiments of the present invention a CMA-12PP linear actuator (Newport) is connected to a 1-mL syringe and run at a rate of 0.0003 MM/s, corresponding to a volumetric flow rate of 0.019 mL/h and a flow velocity of ˜66 mm/s out of the injket nozzle.

The device is built on an modified microscope base to facilitate vesicle production. The stage can be equipped with translation stages to position the entire system (inkjet device, formation chamber, and fixtures) relative to the microscope base for viewing and to insert and position the inkjet device relative to the vesicle formation chamber. A high-speed camera mounted from the underside of the system can be used to visualize the vesicle formation process. In preferred embodiments of the present invention, a TTL signal generated at the instant that the inkjet device is triggered starts recording by a monochrome Photron 1024PCI high-speed camera at a frame rate of approximately 5000 fps. Generally, a 5 X objective is used, which yields a field-of-view of 2 mm×0.6 mm when limiting the region of interest to 600×200 pixels. The device can also include a side-view camera to align the inkjet system and the vesical chamber along the vertical direction.

Planar lipid bilayers can be created at the interface between two aqueous droplets that are initially surrounded by oil containing dissolved lipids and then brought into contact, an approach pioneered by Bayley et al. to study membrane pores. In certain embodiments of the present invention, planar bilayers with controlled lipid composition are prepared by delivering lipid content through the aqueous phase in the form of SUVs (modified protocol from Hwang, et al.). Using a custom-built chamber containing a small volume of oil, two aqueous droplets are separated by a thin divider and SUVs with oil-insoluble lipids are loaded into each droplet. The SUVs diffuse within the droplets and gradually fuse to the oil-water interface of each droplet, forming a continuous lipid monolayer around each droplet. Upon removal of the divider, the two droplets move into contact and create a planar bilayer membrane at the interface. Preferred embodiments of the invention provide asymmetric planar bilayers by incorporating different SUVs into each of the aqueous droplets or by loading SUVs into one droplet and allowing lipid soluble in oil to form the monolayer of the second droplet. The inner leaflet and the outer leaflet can be independently and selectively formed to include membrane proteins and/or functionalized lipids. Planar membranes can also be formed from a combination of lipids dissolved in oil and oil-insoluble lipids added via SUVs to the aqueous droplets. GUVs made from these bilayers will contain both oil-insoluble and oil-soluble lipids, but lack controlled composition. This approach can be used to minimize background SUV concentration, and may be most appropriate for doping in signaling lipids which typically comprise <1% of the total phospholipid content of cellular membranes, but can be present in other amounts.

The methods of the present invention enable the encapsulation of two solutions—one in the jet and one surrounding the jet—within a vesicle. The fluid jet that forms the vesicle entrains the surrounding fluid to constitute the fluid mixture encapsulated during the vesicle formation process. Control over encapsulation efficiency provides for accurate initiation of chemical reactions between the two solutions within the vesicle at the time of formation as well as real-time management of the encapsulation fraction, allowing for variations in concentration during an experiment which are important for biological reconstitutions and small volume reactions.

In some embodiments, the present invention provides a method of forming a vesicle, the method including contacting an aqueous mixture and an oil mixture, wherein the aqueous mixture includes a first lipid, and the oil mixture includes a second lipid, wherein the aqueous mixture or oil mixture also includes at least one bilayer component of a transmembrane protein or a functionalized lipid. Thus, a lipid bilayer forms at the interface of the aqueous mixture and the oil mixture, wherein the interfacial lipid bilayer includes an aqueous mixture lipid layer having the first lipid, and an oil mixture lipid layer having the second lipid, wherein the interfacial lipid bilayer also includes the membrane protein and the functionalized lipid when present. The method also includes pulsing the interfacial lipid bilayer with a fluid mixture from an inkjet, wherein the fluid mixture includes at least one component of a protein, a peptide, an enzyme, an oligonucleotide, or a polynucleotide. Thus, the vesicle is formed.

In other embodiments, the first and second lipids are the same and the aqueous mixture also includes the functionalized lipid, the aqueous mixture lipid layer includes the first lipid and the functionalized lipid, and the oil mixture lipid layer includes the second lipid, thereby forming an asymmetric interfacial lipid bilayer. In some other embodiments, the first and second lipids are the same and the oil mixture also includes the functionalized lipid, the aqueous mixture lipid layer includes the first lipid, and the oil mixture lipid layer includes the second lipid and the functionalized lipid, thereby forming an asymmetric interfacial lipid bilayer.

In still other embodiments, the first and second lipids are the same, and the aqueous mixture or the oil mixture includes the membrane protein. In yet other embodiments, the fluid mixture includes a second component of a protein, a peptide, an enzyme, an oligonucleotide, a polynucleotide, a diagnostic agent or a therapeutic agent.

### VI. EXAMPLES

### Example 1

### Giant Unilamellar Vesicle Formation

This example describes the development of a system capable of simultaneously forming and loading unilamellar vesicles at rates up to 200 Hz using microfluidic inkjet printing. Fluid loaded into a piezoelectric-actuated inkjet is accelerated through a micron-scale nozzle to create a high-speed liquid jet. We take advantage of the precision, capacity for control, and high-frequency displacements inherent to piezoelectric inkjets, a technology originally developed for printing applications. Using this approach we achieve high-throughput vesicle production and control vesicle size over a range of approximately 10 to 400 μm in diameter corresponding to more than four orders of magnitude in volume. Since the vesicles can be imaged immediately after their formation, reaction dynamics and bilayer membrane interactions can be followed from initiation. The ability to rapidly form multiple vesicles of equal size also enables large numbers of experiments to be conducted simultaneously so that statistically significant behavior can be identified. This combination of attributes makes inkjet printing of unilamellar vesicles a simple, robust, and rapid method for generating small reaction volumes with cell-like membrane boundaries.

### Materials and Methods

Reagents. 1,2-Diphytanoyl-sn-glycero-3-phosphocholine (DPhPC) lipid was purchased dissolved in chloroform from Avanti Polar Lipids, resuspended in n-decane (Sigma-Aldrich) at a concentration of 25 mg/ml, and stored at −20° C. with a dessicant (Drierite). Sucrose and glucose were prepared at approximately 400 mOsm. Ficoll PM 400 (Amersham Biosciences) solutions were prepared at 7.5-8.75 weight percent, and 300 mM glucose was added to aid in visualization during jetting.

Preparation of TMR-PIP₂-containing liposomes. DPhPC/TMR-PIP2 liposomes were prepared by the method of sonication. Lipids stored in chloroform were mixed to the following ratio: 99% (DPhPC) (Avanti), 1% BODIPY-tetramethylrhodamine-phosphatidylinositol-4,5-bisphosphate (TMR-PIP₂) (Echelon Biosciences); dried under nitrogen; and desiccated for 90 min. Lipids were rehydrated to a final concentration of 0.5 mg/mL by the addition of 10 mM HEPES pH 7.5, 200 mM KCl for 15 min, briefly vortexed, and then sonicated by a tip sonicator (Sonicator 3000, Misonix). The solution was spun for 20 min at 10,000× g at room temperature and the supernatant was used for experiments.

Preparation of DOGS-NTA-Ni-containing liposomes. DPhPC/DOGS-NTA-Ni liposomes were prepared by the method of sonication. Lipids stored in chloroform were mixed to the following ratio: 95% 1,2-diphytanoyl-sn-glycero-3-phosphocholine (DPhPC) (Avanti), 5% 1,2-dioleoyl-sn-glycero-3-[(N-(5-amino-1-carboxypentyl)-iminodiacetic acid)succinyl] (nickel salt) (DOGS-NTA-Ni) (Avanti); dried under nitrogen; and desiccated for 90 min. Lipids were rehydrated to a final concentration of 2 mg/mL by the addition of 10 mM HEPES pH 7.5, 200 mM KCl for 15 min, briefly vortexed, and then tip sonicated. The solution was spun for 20 min at 10,000× g at room temperature and the supernatant was used for experiments.

Protein expression. GFP-UV was expressed as a His₆-tagged fusion protein (His-GFP) in BL21 (DE3) pLysS cells (Stratagene). Cells were grown at 37° C. until OD₆₀₀ of 0.3-0.5, induced with 40 μM IPTG and grown for 14-16 h at 18° C. Cells were harvested and resuspended in PBS containing 10 mM imidazole, and lysed by freeze-thawing and sonication. The lysate was centrifuged for 45 min at 20,000× g at 4° C., and the supernatant was incubated with 0.5 mL of Ni-NTA Agarose beads (Quiagen) per 1L of culture for 1-2 h. Beads were washed 7 times with PBS containing 10 mM imidazole and twice for 15 min in the same buffer. The protein was eluted with PBS containing 150 mM imidazole, dialyzed into PBS, concentrated, aliquoted, and frozen.

Rheometry. Viscosity of solutions was measured at low shear rates (18-50 s⁻¹) using a Gemini rheometer (Malvern Instruments) with a concentric cylinder geometry.

Planar bilayer lipid membranes. Vesicles were formed from planar lipid bilayer membranes constructed by contacting monolayers in a double-well chamber. In some embodiments, bilayers were formed at the intersection of chamber wells (30-80 μL each), where chambers had the shape of an “infinity” symbol, and the bilayer was formed at the waist of the pattern, However, chamber wells of arbitrary shape are possible, where circular, ellipsoidal, and rectangular shapes have been used. Chambers were made using a laser cutter (Versa Laser) typically from 3 mm acrylic sheets (TAP plastics) and bonded to acrylic cover slips (0.2 mm thick, Astra Products) using acrylic cement (TAP plastics). A hole of approximately 1.5 mm diameter was drilled on one end of the chamber, perpendicular to the bilayer plane, to facilitate insertion and positioning of the inkjet nozzle. Adhesive (Locktite 495) adhered a thin (0.26 mm) sheet of natural rubber (McMaster-Carr) to the external surface of this hole to provide a seal. A needle was used to make a small hole in the rubber sheet for insertion of the inkjet nozzle. To form bilayer lipid membranes, approximately 12 μL of lipid solution (25 mg/mL DPhPC dissolved in n-decane) was pipetted into the chamber. Aqueous droplets were pipetted sequentially into each of the fluid chambers and rapidly came into contact, forming the bilayer lipid membrane at their interface.

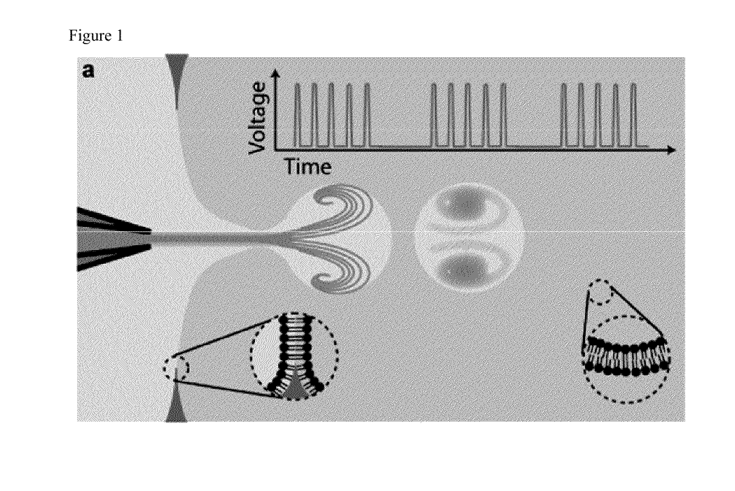

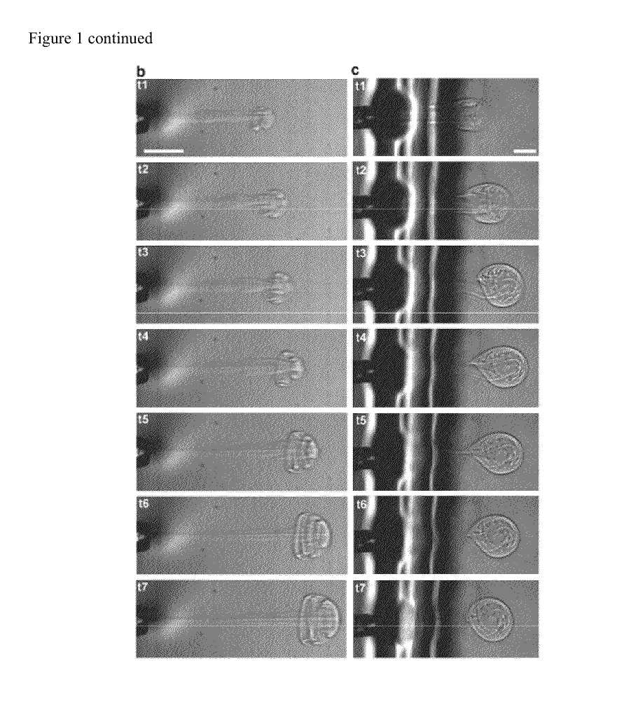

In other embodiments, chambers were designed with two cylindrical bores (4.3 mm diameter) separated by a 0.5 mm wide slot through the center that holds a thin acrylic divider. When the divider is removed, it leaves a 3 mm wide ‘window’ between the two cylinders. Chambers were cut from sheets of 4.5 mm thick acrylic (McMaster-Carr) using a laser cutter (Versa Laser). A 1.5 mm hole was drilled in one end of the chamber, and covered by a 0.26 mm latex film (McMaster-Carr), forming a seal. A small hole was made in the latex film with a 23G needle to allow insertion and alignment of the inkjet nozzle (FIG. 1a). Finally, a thin (0.2 mm) acrylic coverslip (Astra products) was cemented (acrylic cement, TAP plastics) to the bottom of the chamber. Chambers were cleaned with 2% Neutrad (Decon Laboratories) solution at 60° C., thoroughly rinsed and re-used multiple times. A planar bilayer was prepared by incubating two SUV containing droplets (45 μl each), separated by a thin (0.2 mm) acrylic divider, in an oil (decane, 40 μl total) loaded acrylic chamber. Incubation was typically done overnight at 4° C. However, when forming asymmetric (protein-free) lipid bilayers, the incubation time was reduced to tens of minutes to prevent trans-bilayer mixing of lipids by diffusion through the oil. SUV concentrations in the droplets ranged from 0.02-0.5 mg/ml. Typically, if 0.1 mg/ml or lower SUVs were used, 30 μl of 50 mg/ml DPhPC was added to the oil to a final concentration of 21 mg/ml shortly before bilayer formation to improve stability of the bilayer (this was unnecessary when higher SUV concentrations were used). Bilayer formation was initiated by removal of the thin acrylic divider. Bilayers were typically stable for more than 1 hour.

Inkjet device and vesicle formation. Inkjets and drive electronics were from MicroFab Technologies. Inkjets used home built glass orifices installed by MicroFab. Orifices were made from glass capillary stock (0.6 mm inner diameter and 0.75 mm outer diameter) and formed by pulling the capillaries into micropipettes with sharp tips (P97 Flaming/Brown Micropipette Puller, Sutter Instruments). Micropipettes were refined using a Microforge MFG-3 (MicroData Instruments) and sanded to the desired orifice inner diameter, 10 μm. Disposable syringes were used to load inkjets with the jetted solution from the rear and provided constant perfusion of this solution (approximately 10 μL/min) during experiments. Loaded inkjets were inserted into bilayer lipid membrane chambers and positioned with less than 200 μm between the nozzle orifice and bilayer. Multiple fluid pulses from the print head (12-35) were used to form vesicles. Application of a trapezoidal voltage pulse from the drive triggered each pulse. Identical pulses repeated at 20 kHz had a rise time of 3 μs, amplitude of 25-35 V, duration at this amplitude of 35 μs (to maximize velocity), and fall time of 3 μs. Vesicle formation was recorded using high-speed microscopy (500-30,000 frames per second, Photron 1024 PCI). A wide range of solutions were encapsulated in GUVs using this technique, but the encapsulated solution was always osmotically balanced to the inner and outer droplet solutions, and typically contained >100 mOsm sucrose to achieve optical contrast and sink the GUV for ease of imaging. Vesicle formation was recorded using a high-speed camera (1024PCI, Photron). After formation, GUVs were assessed for the presence of entrained oil. Oil lenses were easily identified by increased signal from fluorescently labeled lipids or the exclusion of transmembrane proteins in this domain (FIG. S3), and can also be identified by phase contrast differences using phase microscopy. GUVs containing significant oil were excluded from our analysis. In order to characterize success rate of vesicle formation, we recorded the fraction of bilayers from which vesicles were made, for each of five consecutive days of experiments. On average, vesicles were formed each day from 66% +/−13% (s.e.m.) of bilayers (n=30 bilayers).

Incorporation of TMR-PIP₂ into GUVs. A thin acrylic divider was placed in a 4.5 mm-deep acrylic chamber with 6 mm diameter bores and the chamber was loaded with 100 μL decane(Sigma-Aldrich). 90 μL droplets containing 0.1 mg/ml DPhPC/TMR-PIP₂ liposomes were added to both sides of the chamber, and the chamber was incubated overnight at 4° C. The thin acrylic divider was removed, and the chamber was left for 5-10 minutes to allow for planar bilayer formation before an inkjet loaded with 350 mOsm sucrose was used to form GUVs by microfluidic jetting.

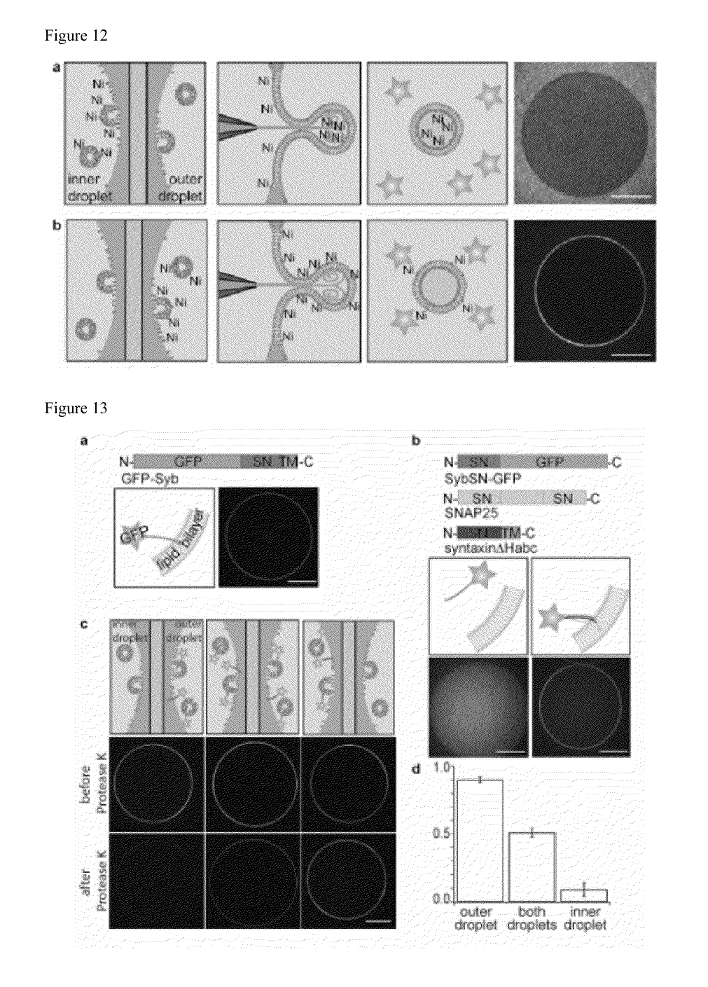

Asymmetric incorporation of DOGS-NTA-Ni into GUVs. DOGS-NTA-Ni was selectively incorporated into the inner leaflet of a GUV. First, a thin acrylic divider was placed in a 3 mm-deep acrylic chamber with 6 mm diameter bores and the chamber was loaded with 20 μL of 25 mg/ml DPhPC in decane. 120 μL of 1× PBS was added to the outer droplet, 120 μL of 0.02 mg/ml DPhPC/DOGS-NTA-Ni SUVs was added to the inner droplet, and the chamber was incubated for 15 min (FIG. 12a). The divider was removed, and a planar bilayer formed. An inkjet containing 100 mOsm sucrose in 0.875× PBS was inserted into the chamber and used to form GUVs from the planar bilayer. After vesicle formation, His-GFP was added to a final concentration of 0.311 M in the final droplet, and incubated for 30 min before images were captured by confocal microscopy (FIG. 12a). DOGS-NTA-Ni was selectively incorporated in the outer leaflet of a GUV by repeating this procedure, but the outer droplet was 120 μL 0.02 mg/ml DPhPC/DOGS-NTA-NiSUVs and the inner droplet was 120 μL 1× PBS (FIG. 12b).

Alternatively, DOGS-NTA-Ni was incorporated into the inner leaflet of a GUV by setting up the oil-containing chamber with an inner droplet containing 0.02 mg/ml DPhPC/DOGS-NTA-Ni SUVs and an outer droplet containing 0.02 mg/ml DPhPC SUVs, and was incubated for one hour. After incubation, DPhPC in oil was added to a final concentration of 21 mg/ml in the chamber. The thin acrylic divider was removed and GUVs were formed using an inkjet containing either 2 μM His-GFP and 6% iodixanol in 10 mM Hepes pH 7.5, 200 mM KCl, or only 6% iodixanol in 10 mM Hepes pH 7.5, 200 mM KCl. After formation of multiple GUVs from a single bilayer, the sample was transferred to a spinning disc confocal microscope for imaging. GUVs with encapsulated His-GFP were imaged immediately. 0.3 μM His-GFP was added to chambers with GUVs lacking encapsulated His-GFP, and they were incubated for 30 min before imaging. Selective incorporation of DOGS-NTA-Ni into the outer leaflet of a GUV was accomplished by repeating this procedure, with 0.02 mg/ml DPhPC/DOGS-NTA-Ni SUVs in the outer droplet and 0.02 mg/ml DPhPC SUVs in the inner droplet.

Asymmetric incorporation of DPPE-NBD into GUVs. DPPE-NBD was selectively incorporated into the outer leaflet of a GUV. A thin acrylic divider was placed in a 3 mm-deep acrylic chamber with 6 mm diameter bores and the chamber was loaded with 20 μL of 25 mg/mL DPhPC in decane. 120 μL of 0.2 mg/mL DPhPC/DPPE-NBD liposomes was added to the outer droplet, 120 μL of 200 mM KCl, 10 mM HEPES pH 7.5 buffer was added to the inner droplet, and the chamber was incubated for 15 min. The divider was removed, and a planar bilayer formed. An inkjet containing 350 mOsm sucrose was inserted into the chamber and used to form GUVs from the planar bilayer. Images were taken with confocal microscopy before and 10 min after exposure to 10 mM sodium dithionite, pH 10.1.

The fluorophore N-(7-nitro-2-1,3-benzoxadiazol-4-yl) (NBD) is irreversibly quenched when exposed to the membrane impermeable reducing agent, sodium dithionite (Sigma-Aldrich). Thus, the fraction of an NBD-labeled lipid in the external leaflet of a vesicle (Fₒᵤₜ) can be measured from the ratio of fluorescence intensity before (I_{before}) vs. after (I_{after}) addition of sodium dithionite.

Quantification of DPPE-NBD fraction in the outer leaflet of the GUV was carried out using ImageJ. A line scan was taken of the ‘before’ image, and the fluorescence intensity of the membrane was measured from which the background fluorescence intensity was subtracted. This was repeated for the ‘after’ image, and fₒᵤₜ=0.94 +/−0.03 (s.e.m., n=4 bilayers) was calculated.

We used the head-labeled lipid 1,2-dipalmitoyl-sn-glycero-3-phosphoethanolamine-N-(7-nitro-2-1,3-benzoxadiazol-4-yl) (DPPE-NBD) (Avanti) because we found that the tail-labeled lipid 1-palmitoyl-2-(6-[7-nitro-2-1,3-benzoxadiazol-4-yl)amino]hexanoyl)-sn-glycero-3-phosphocholine (Avanti) did not have sufficient solvent exposure to be quenched in the planar geometry of a GUV (data not shown).

Results and Discussion

Formation of single unilamellar lipid vesicles with multiple pulses of an inkjet. We used a drop-on-demand inkjet to produce a microfluidic jet that deforms a planar bilayer lipid membrane into unilamellar vesicles that separate from the planar membrane (FIG. 1a). The inkjet generates multiple discrete pulses using a cylindrical piezoelectric actuator surrounding a fluid filled nozzle. The actuator contracts and expands radially, producing pressurization and rarefaction waves in the fluid. Application of appropriate voltage pulses to such devices results in the ejection of fluid from the device due to the constructive interference of traveling pressurization waves (and destructive interference of traveling rarefaction waves) within the nozzle. In our work, multiple, closely spaced (20 kHz) voltage pulses applied to the inkjet produced a set of fluid pulses. As they traveled in and entrained the surrounding fluid, these fluid pulses combined to form a growing vortex ringstructure (FIG. 1b) that was capable of deforming the bilayer lipid membrane to form vesicles (FIG. 1c). Examination of selected frames from a high-speed video (22,500 frames per second) recorded during jetting shows the formation and addition of individual fluid pulses into a composite vortex ring (FIG. 1b). Vesicle formation occurs via a process similar to the one we observed for a single pulse jet flow generated by a piezo-actuated syringe. Briefly, membrane deformation (t1, FIG. 1c), membrane collapse (t2-t4, FIG. 1c), and vesicle separation (t5-t6, FIG. 1c) stages were observed over about 5 ms, typically. Vesicles formed were highly monodisperse (for example 194.1 μm±4.6 μm average diameter and first standard deviation, N=50 vesicles formed consecutively). Vesicles contain a mixture of the fluid loaded in the inkjet and the fluid surrounding the inkjet, which is entrained as the vortex ring forms and grows. Isolated vortex rings have been shown to contain 25-40% entrained volume. We expect a somewhat lower fraction of entrained fluid to be encapsulated inside vesicles because the vortex ring is partially confined by the lipid membrane as it grows.

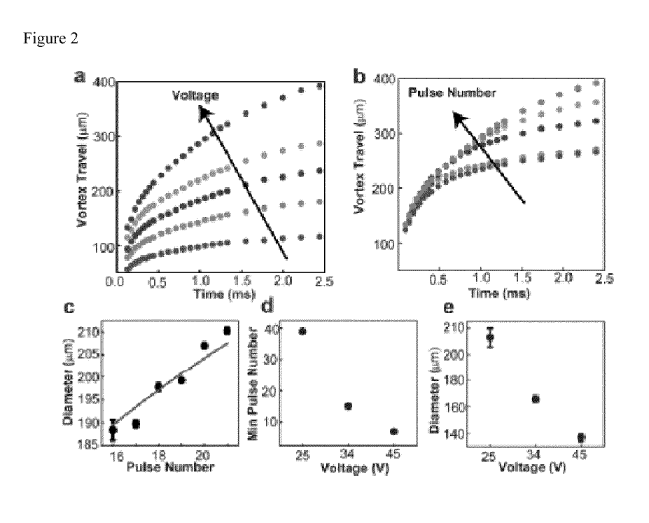

Control of vesicle formation using inkjet parameters. Use of an inkjet to form vesicles with multiple pulses provides the opportunity to control the vesicle formation process by tuning multiple inkjet parameters. Two inkjet parameters expected to directly influence the resulting vesicle formation process are (i) the pulse amplitude and (ii) the number of pulses used to form each vesicle. We expect these parameters to correlate strongly with the vortex velocity and volume, respectively. We began by investigating the impactof pulse amplitude and number on the initial motion of the vortex ring leading edge, a property of the jet that can be conveniently measured by video microscopy (FIG. 2a-b). In all cases, the travel velocity decayed rapidly due to the effects of fluid entrainment and drag. When the pulse amplitude was increased with fixed pulse number, the primary result was an increase in vortex ring travel velocity (increase in slope of curves in FIG. 2a). In contrast, when the number of pulses increased at constant pulse amplitude, the initial travel velocity (initial slope of curves in FIG. 2b) remained roughly constant while the duration of travel increased. In this way, pulse amplitude appears to control the vortex travel velocity, while pulse number appears to control the duration of travel.

We next examined the impact of pulse amplitude and number on the vesicle diameter. We held the pulse amplitude at 35 volts and varied the pulse number at fixed separation between the bilayer and nozzle (FIG. 2c). Vesicles were first formed using 16 pulses. We found that the vesicle diameter increased monotonically with increasing number of pulses from approximately 187 μm (16 pulses) to 210 μm (21 pulses). The relationship between pulse number and vesicle diameter was fit reasonably well (R²=0.917, N=18) by a power law relation with exponent ⅓, as would be expected if the vesicle volume varied proportionally with pulse number. However, the ideal fit (R²=0.965) was achieved with a somewhat larger exponent (0.429±0.04) indicating that vesicle volume increased with pulse number faster than could be accounted for by a linear relationship. This additional growth likely arose from fluid entrainment, which had more time to evolve as the pulse number increased, increasing the duration of the composite pulse. We examined the combined impact of variations in pulse number and amplitude on vesicle diameter, identifying the minimum number of pulses needed to create a vesicle, 7-39, over a range of pulse amplitudes, 25-45 volts (FIG. 2d). We found that the minimum number of pulses decreased with increasing pulse amplitude, and the vesicle diameter decreased with increasing pulse amplitude (FIG. 2e) from approximately 210 to 140 μm. Here as pulse velocity increased with increasing amplitude, the amount of membrane deformation achieved by each pulse increased such that fewer pulses were needed to form a vesicle, and the resulting vesicle was smaller.

Formation of cell-sized vesicles through variation of fluid viscosity. Some experiments using small encapsulated volumes, particularly those aimed at reconstituting specific cellular processes, would benefit from vesicle dimensions on the scale of typical single cells. Vesicle diameters in the range of 1-50 μm would better emulate most eukaryotic and some bacterial cells, extending the range of encapsulation applications. Since vesicle volume is defined during the encapsulation process by collapse of the deformed membrane around the jetted fluid,we hypothesized that differences in viscosity on either side of the bilayer membrane would alter the collapse dynamics and resulting vesicle size. First, we found that jetting of a more viscous solution (8.75 w/v % ficoll 400 and 300 mM sucrose dissolved in water ˜5 cp) while maintaining an inviscid solution on both sides of the bilayer (300 mM glucose dissolved in water ˜1 cp) led to a dramatic increase in vesicle diameter (380 μm diameter, data not shown).

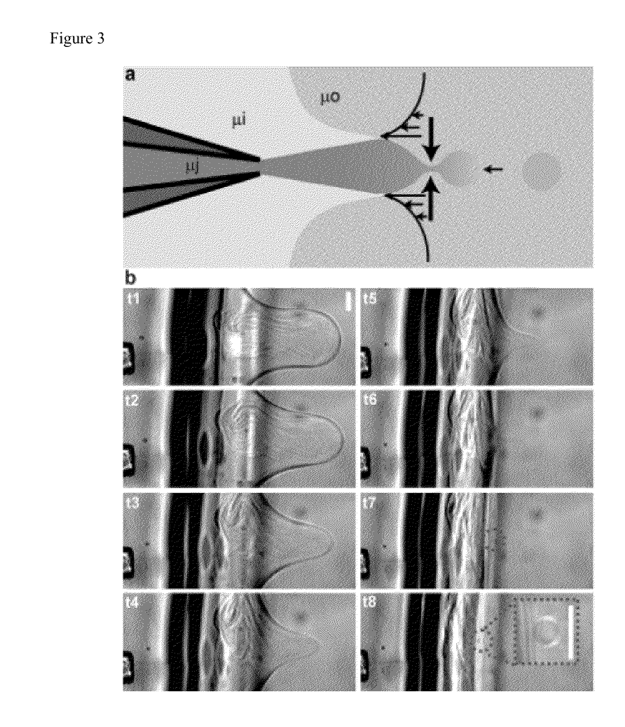

Next, we investigated whether jetting a less viscous fluid into a more viscous fluid would lead to the formation of a smaller vesicle. The fluid in the jet (300 mM sucrose solution) and on the jet side of the bilayer (300 mM glucose solution) had a viscosity of approximately 1 cp, while the viscosity of the fluid on the opposing side of the bilayer had approximately 3.9 cp viscosity (7.5 w/v % ficoll 400 and 300 mM glucose solution) at low shear rates as measured by rheometry (FIG. 3a). We observed that vesicles of significantly smaller size (approximately 8-110 μm) were formed in this configuration (FIG. 3b). Notably, vesicles were formed during retraction of the membrane rather than during expansion, and the process of formation was characterized by an asymmetric retraction, not observed in the absence of a viscosity differential. Specifically, lateral retraction of the membrane sides occurred more rapidly than axial retraction of the membrane, such that the aspect ratio (axial extent to lateral extent) of the membrane protrusion increased during retraction until a vesicle was formed.

Vesicle formation is successful when the lateral retraction of membrane sides comes to completion before the axial retraction of the membrane, allowing a portion of the membrane area to separate. The observered decrease in vesicle size with increase in viscosity differential is expected considering that each membrane retraction process, axial and lateral, depends on viscosity. Axial retraction involves dragging the vesicle bolus through the surrounding fluid and is therefore slowed down by increasing the viscosity of the fluid on the side of the bilayer opposite the jet. Lateral retraction requires expulsion of the encapsulated fluid through the bolusneck and is thus sped up by decreasing the viscosity of the fluid on the jet side of the bilayer (in and around the jet). Therefore, when a relatively higher viscosity solution is used in the jet, the rate of axial retraction increases relative to the rate of lateral retraction such that longer, more powerful ejections are required to form vesicles, and the resulting vesicles are larger. In contrast, use of a lower viscosity in the jet than on the opposing side of the membrane decreases the rate of axial retraction in comparison to lateral retraction, resulting in an asymmetric contraction of the membrane that enables formation of smaller vesicles. Cell-sized vesicles formed using this viscosity differential were monodisperse with a typical variation in diameter of approximately 10% within populations made using the same jetting parameters (relative viscosity, voltage, pulse number, and nozzle-bilayer separation). It is likely that these parameters can be varied in order to maintain a specific vesicle size while accommodating variations in the composition and viscosity of encapsulated solution.

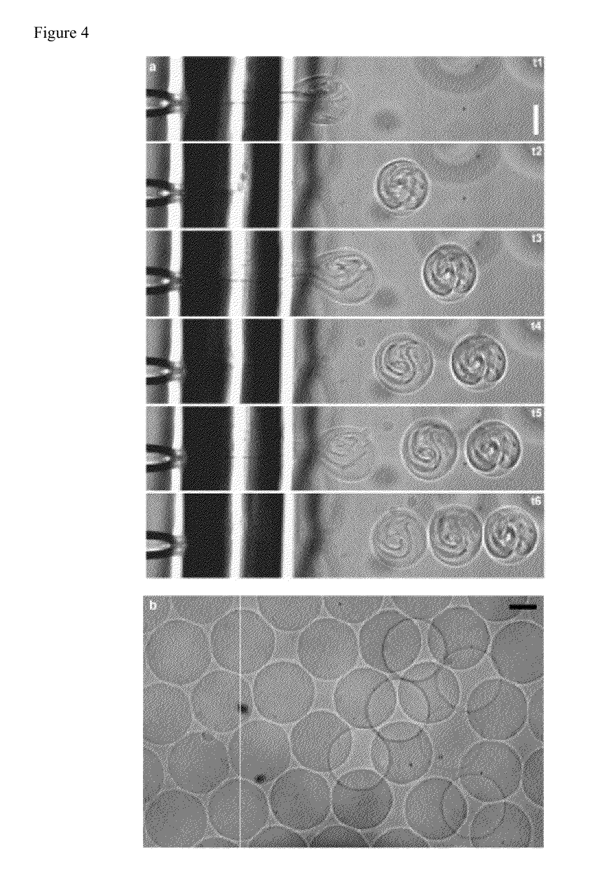

High-throughput inkjet formation of vesicles. Use of an inkjet enables rapid production of large numbers of vesicles, facilitating statistical studies of system behavior and emergent properties, similar to the study of a population of cells. Analogous to the continuous production of ink drops during document printing, high-throughput formation of vesicles takes advantage of the high bandwidth of piezoelectric actuators. Each vesicle is formed by a set of closely spaced (20 kHz) pulses and the temporal spacing of pulse sets determines the rate of vesicle formation. We investigated vesicle formation at rates from 0.5 Hz to 200 Hz (FIG. 4), seeking to understand the limitations on formation rate.

At low formation frequencies, we found that continuous vesicle formation was possible. At a rate of 0.5 Hz, the inkjet printing method was able to consistently form hundreds of vesicles. Cell-sized vesicles formed using a viscosity differential across the bilayer were also produced at this rate. Upon increasing the rate of vesicle formation above 1 Hz, we found that some of the supporting oil matrix was included in the vesicle boundaries after 10-15 solvent-free vesicles were formed. It is interesting to note that the vesicles formed before oil was observed have a total surface area of the same order as that of the bilayer lipid membrane (˜1 mm²) Based on this observation it appears likely that the inclusion of oil in the vesicle boundaries after 10-15 vesicles were formed indicates that the rate of bilayer membrane regrowth from the oil phase was not keeping up with the rate of membrane removal at formation rates above 1 Hz. At higher frequencies, multiple solvent-free vesicles could still be formed, though oil fouled the vesicles after only a few (less than 15) were made. When small batches of vesicles are formed, the rate of membrane regrowth no longer limits the vesicle production rate. Instead, the formation rate should be limited by the time required for a single vesicle to form and the lipidmembrane to relax back to its original position, which is about 5 ms. In agreement with this idea, we were able to form several solvent-free vesicles at rates up to 200 Hz (FIG. 4a). After several vesicles are formed at this rate, a pause in vesicle formation of 2-4 seconds allows the bilayer to regrow. In this way, multiple batches of several solvent-free vesicles each were formed with an inter-batch spacing of 2-4 seconds and an inter-vesicle spacing of as little as 5 ms (200 Hz). This maximum rate of vesicle formation may be particularly useful if the rate of membrane regrowth can be increased such that it is no longer limiting, and it therefore likely represents an upper limit for vesicle production rates.

Formation of vesicles with controlled lipid composition. A first step towards engineering systems that recapitulate the physical boundary conditions of cells is encapsulation of components in giant vesicles of controlled lipid chemistry. Several recent microfluidic techniques have achieved formation of giant vesicles with controlled contents using oil-water interfaces to define lipid membranes. However, these techniques form the membranes from lipids dissolved in oil and are incompatible with many biologically important lipids that display poor solubility in oil due to their net charge or saturated fatty acid tails. For example, the lipid phosphatidylinositol-4,5-bisphosphate (PIP₂) is involved in many essential signaling pathways but displays poor solubility in oil due to its strong negative charge.

In order to form giant vesicles with controlled contents, we first assembled a planar bilayer and then generated GUVs by microfluidic jetting. Planar lipid bilayers can be created at the interface between two aqueous droplets that are initially surrounded by oil containing dissolved lipids and then brought into contact. We have previously demonstrated that these planar bilayers can be deformed by microfluidic jetting with a piezo-electric inkjet nozzle to form giant unilamellar vesicles. However, our previous use of lipids dissolved in oil prevented the inclusion of physiologically important signaling lipids.

To overcome this limitation, we formed planar bilayers with controlled lipid composition by delivering lipid content through the aqueous phase in the form of SUVs. Using a custom acrylic chamber containing a small volume of oil, we kept the two aqueous droplets separated by a thin divider and loaded SUVs with the oil-insoluble lipid of interest into each droplet. The SUVs diffused within the droplets and gradually fused to the oil-water interface of each droplet, forming a continuous lipid monolayer around each droplet with lipid composition defined by the lipid in the SUVs rather than lipid dissolved in the decane (FIG. 11a). When the divider was removed, the two droplets moved into contact, excluded oil from the contact region between the droplets, and formed a large (˜2 mm²) planar bilayer from the closely opposing lipid mono layers (FIG. 11b). Giant unilamellar lipid vesicles with controlled lipid composition were formed from this planar bilayer by microfluidic jetting (FIG. 11c).

We used this procedure to incorporate fluorescently labeled PIP₂ (TMR-PIP₂) into giant vesicles (FIG. 11d). Confocal microscopy confirmed PIP₂ delivery to the GUV membrane. We also tested the generality of this protocol by incorporating functionalized lipids with large poly(ethylene glycol) (PEG) chains (1,2-distearoyl-sn-glycero-3-phosphoethanolamine-N-[PEG2000-N′-carboxyfluorescein]) and charged Ni-chelating head groups (1,2-dioleoyl-sn-glycero-3-[(N-(5-amino-1-carboxypentyl)-iminodiacetic acid)succinyl]) (DOGS-NTA-Ni). Both of these lipid species exhibit poor solubility in oil.

Formation of GUVs with asymmetric membranes. In addition to complex composition, cellular membranes are generally asymmetric in nature, maintaining a different environment on their cytosolic and extracellular sides. In order to mimic this fundamental feature of cellular membranes, we independently controlled the lipid composition in each leaflet of the unilamellar vesicle. Asymmetric planar bilayers can be formed as described above by incorporating different SUVs into each of the aqueous droplets or by loading SUVs into one droplet and allowing lipid soluble in oil to form the monolayer of the second droplet. We formed giant vesicles with asymmetric membranes from the asymmetric planar bilayers again by microfluidic jetting. Since the continuity of the membrane is maintained during the vesicle formation process, the internal leaflet of the GUV originated from the lipid monolayer coating the droplet nearest the inkjet nozzle (inner droplet), and the external leaflet of the GUV came from the lipid monolayer coating the far droplet (outer droplet) (FIG. 12). In this way, inner and outer leaflet composition could be independently defined.

We demonstrated this method for formation of GUVs with asymmetric membranes by selectively incorporating oil-insoluble Ni-chelating lipids into the inner or outer leaflet of the bilayer. We confirmed the location of the Ni-chelating lipids by adding 6×-His tagged Green Fluorescent Protein (His-GFP), which has a high affinity for Ni, to the surrounding solution after vesicle formation. When Ni-chelating lipids were added only to the inner leaflet of the GUVs, His-GFP was observed to remain in solution (FIG. 12a). However, when Ni-chelating lipids were incorporated only into the external leaflet of GUVs, His-GFP was seen to strongly associate with the GUV membrane (FIG. 12b), confirming the selective incorporation ofNi-chelating lipids into the outer leaflet of the GUV.

Membrane asymmetry was also confirmed by an independent, quantitative fluorescencequenching assay. For this assay, we selectively incorporated 1,2-dipalmitoyl-sn-glycero-3-phosphoethanolamine-N-(7-nitro-2-1,3-benzoxadiazol-4-yl) (DPPE-NBD) into the outer leaflet of a GUV and measured the fluorescence intensity of the GUV membrane before and after addition of the quenching agent sodium dithionite. The fraction of DPPE-NBD lipids in the external leaflet was calculated from the ratio of the final to initial fluorescence intensity of the GUV membrane, and the analysis revealed that 93+/−4% (s.e.m., n=4 bilayers) of the DPPE-NBD was localized in the external leaflet by this technique (see online Methods for details ofanalysis). While flipping of lipids from one leaflet to the other can occur over long times (many hours to days), we did not observe mixing of the monolayers over the short timescale (˜1 hour) of our experiments.

### Example 2

### Actin Network Polymerization Within GUVs

To demonstrate how inkjet formation of vesicles can be used to create and control biological reactions, we loaded giant unilamellar vesicles with purified monomeric actin and tracer particles and initiated actin polymerization within the vesicles during encapsulation. Immediately after formation of the vesicles, we observed the dramatic increase in particle confinement that accompanied the actin network assembly into an entangled network. Cellular reconstitution studies such as these, along with pharmaceutical challenges that require encapsulation of multiple components, will benefit from inkjet formation of unilamellar vesicles as a platform for discovery.

Materials and Methods

Actin was purified from rabbit muscle acetone powder, and purity was confirmed by sodium dodecyl sulfate polyacrylamide gel electrophoresis. Actin concentration was determined by UV absorbance and BCA assays. To polymerize 9.2 μM actin in the lumen of GUVs (assuming 50% dilution during encapsulation), the inkjet was loaded with 385 mOsm sucrose, 5 mM Tris HCl pH 7.8, 0.2 mM CaCl₂, 0.01% NaN₃, 2 mM ATP pH 7.5, 18 μM actin, and a 300× dilution of 2.63% solids-latex Fluoresbrite Polychromatic red 0.5-μm diameter microspheres (Polysciences, Inc.). Microspheres were sonicated for 5 minutes prior to use in order to break up aggregates. The solution was filtered prior to addition of the microspheres, ATP, and actin. A planar lipid bilayer membrane was set up as described above, and the solution in the droplets surrounding the membrane contained 200 mM KCl, 4 mM MgCl₂, and 2 mM EGTA pH 7.5. The vesicles were formed at 22V and 18 pulses, and bead motion was monitored between 15 and 40 minutes from the time of encapsulation. For the control experiment used to assess microsphere diffusion in the absence of an actin network, solutions A and B were prepared. Solution A contained 385 mOsm sucrose, 5 mM Tris HCl pH 7.8, 0.2 mM CaCl₂, 0.01% NaN₃, and a 333× dilution of sonicated 2.63% solids-latex Fluoresbrite Polychromatic red 0.5-μm microspheres. Solution B contained 200 mM KCl, 4 mM MgCl₂, and 2 mM EGTA pH 7.5. Solutions A and B were mixed in a 1:1 ratio and added to a homemade chamber (0.1 mm×5 mm×20 mm) consisting of a glass coverslip adhered to a glass slide using double-sided tape, then sealed with VALAP (1:1:1 of vaseline, lanoline, and paraffin) before imaging. Bead diffusion was recorded at 100× using a CoolSnap HQ Camera (Photometrics). Bead tracking was done using the Track Points application in Metamorph v7.1 (Molecular Devices), and diffusion curves were calculated using MatLab software (MathWorks).

Results and Discussion

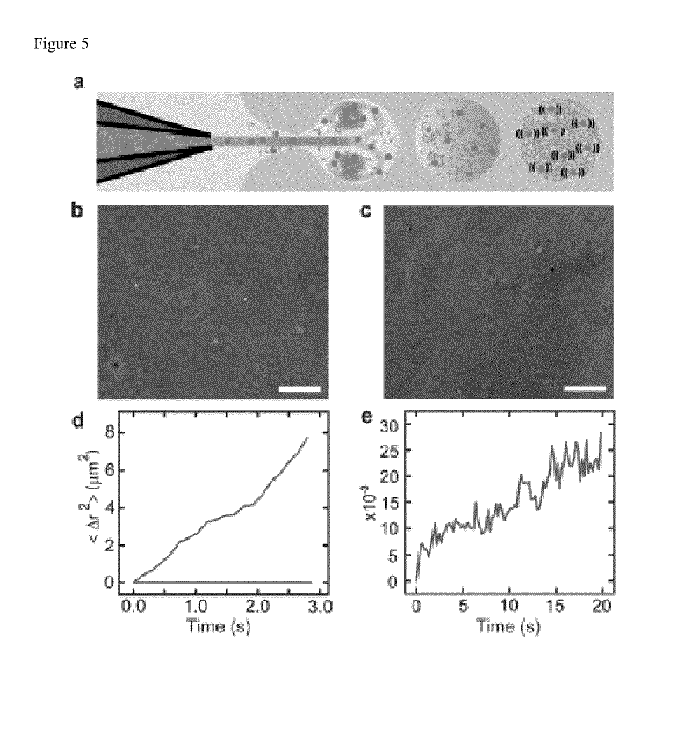

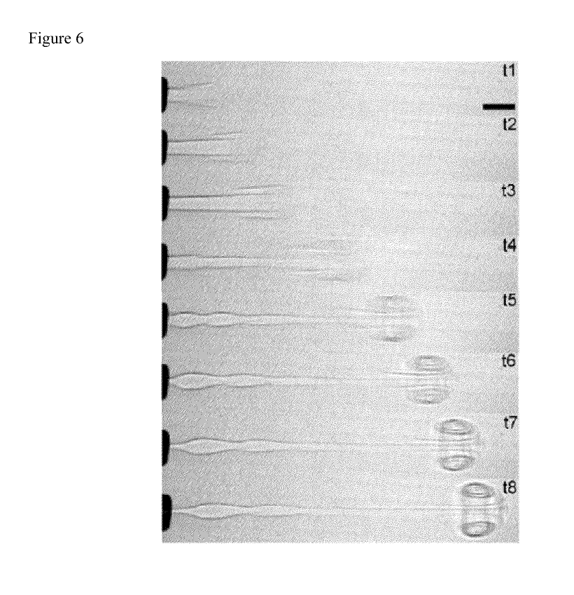

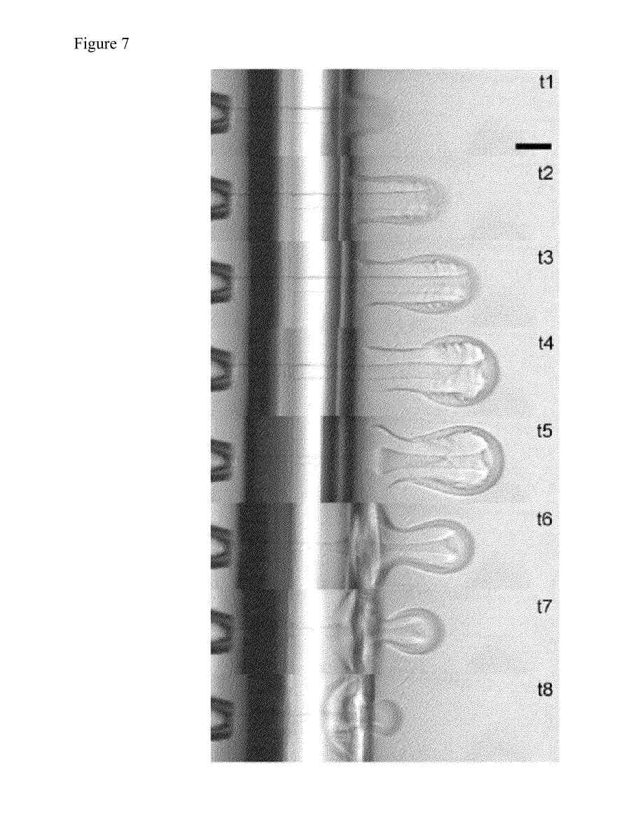

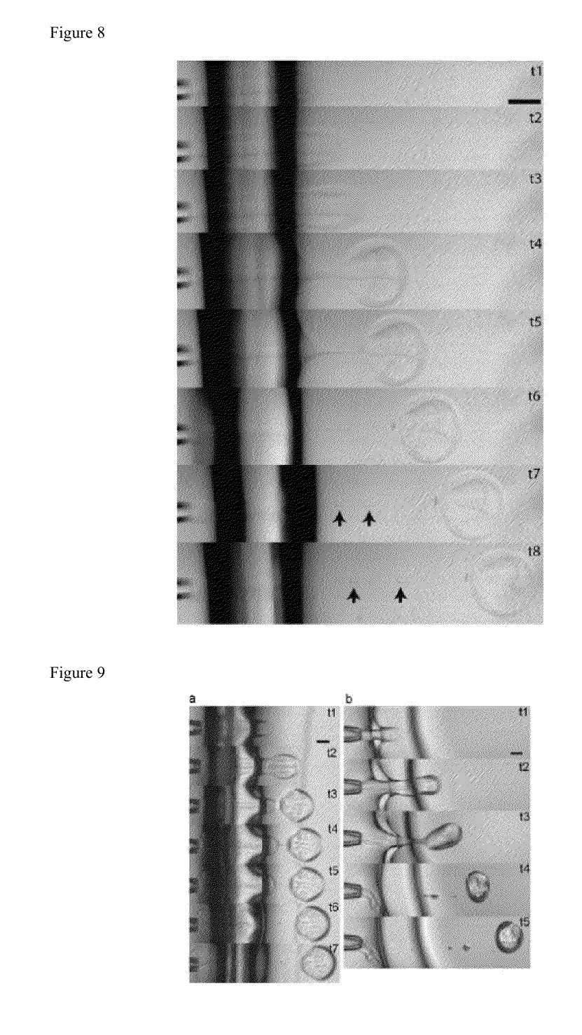

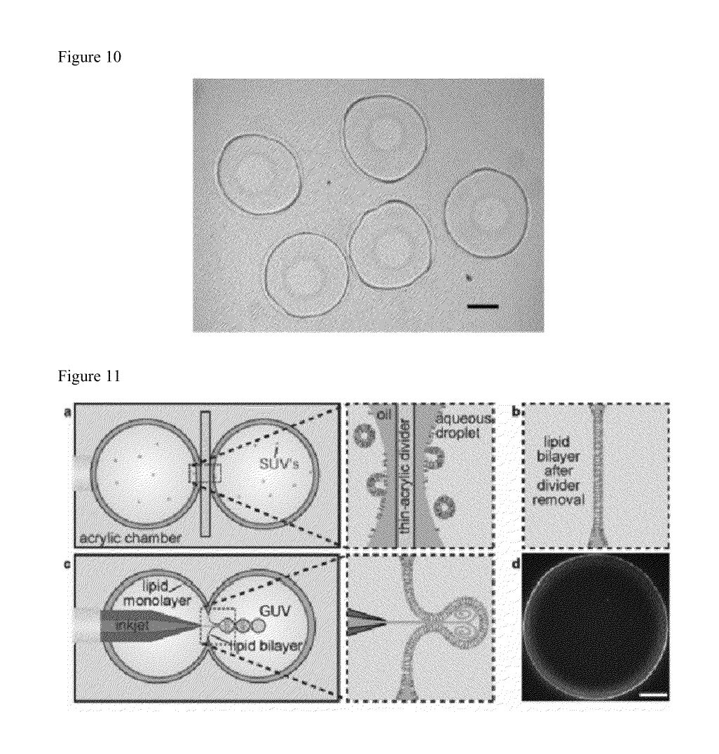

Encapsulation of biological molecules with defined composition and concentration is a significant technical challenge that has limited progress on reconstituting cellular processes in vitro. To demonstrate the utility of vesicle formation and loading by inkjet printing for reconstitution experiments, we loaded and polymerized the cytoskeletal protein actin in the lumen of a GUV (FIG. 5). A bilayer lipid membrane was set up with actin polymerization buffer on each side. Purified actin and latex beads (used to probe solution rheology) were loaded into the inkjet nozzle in monomeric form, and a vesicle containing a mixture of actin monomers (˜0.39 mg/mL monomeric actin), beads, and polymerization buffer was formed (FIG. 5a). Entrainment of actin polymerization buffer during vesicle formation initiated actin polymerization only in the lumen of the GUV, avoiding clogging the inkjet nozzle and temporally defining the start of the reaction such that actin polymerization could be observed immediately following encapsulation.

At long times after vesicle formation (>20 minutes afterward), we observed that all of the encapsulated beads displayed restricted diffusion (FIG. 5c), consistent with the expectation that this concentration of actin monomers should form an entangled network with a mesh size of 0.48 μm, comparable to the bead diameter of 0.49 μm. To confirm that this observation was the direct result of actin polymerization, the sample was illuminated with an arc lamp, causing actin to depolymerize due to damage from reactive oxygen species generated as a result of photobleaching of the microspheres. After 500 ms of exposure, all of the beads were observed to begin diffusing freely again (data not shown). Furthermore, free diffusion of latex beads was observed in the absence of actin monomers (FIG. 5b) and clearly differed from the restricted motion of the encapsulated beads (FIG. 5d-e). Specifically, the slope of the mean squared displacement versus time curve for freely diffusing beads (FIG. 5d, top curve) is more than 3 orders ofmagnitude larger than the slope of the curve for vesicle-encapsulated beads restricted by entangled actin filaments (FIG. 5d, bottom curve, and FIG. 5e). This example demonstrates the ability of microfluidic vesicle formation to perform controlled encapsulation of active protein solutions and initiate biomolecular reactions inside lipid vesicles. Further, controlled encapsulation and polymerization of actin within a unilamellar lipid vesicle is an important step toward reconstitution of the cytoskeleton and its membrane interactions.

### Example 3

### Materials and Methods for Engineering Environmental Sensitivity in an Artificial Cell

The following are the materials and methods used in the following Examples 3-5.

Protein expression. eGFP-rat synaptobrevin (NP_{—}036795, GFP-Syb) was expressed as a His₆-tagged fusion protein in BL21(DE3) pLysS cells (Stratagene). Cells were grown at 37° C. until OD₆₀₀ of 0.9-1, induced with 40 μM IPTG, and grown for 14-16 h at 18° C. Cells were harvested and resuspended in 25 mM HEPES pH 7.5, 400 mM KCl, 5% Triton X-100, 2 mM MgCl₂, 1 mM β-mercaptoethanol (bMe), EDTA-free Complete protease inhibitors (Roche), and DNaseI. Cells were lysed by freeze thawing and the lysate was centrifuged for 45 min at 125,000× g, 4° C. Supernatant was incubated with 2 mL of Ni-NTA Agarose beads (Qiagen) per 1L of culture for 2 h. Beads were washed 7 times with 25 mM HEPES pH 7.5, 400 mM KCl, 1% Triton X-100, 1 mM bMe. 3 more wash steps were conducted with increasing concentrations of imidazole up to 25 mM. Beads were washed 5 times with 25 mM HEPES pH 7.5, 100 mM KCl, 10% glycerol, 1% w/v OGP, 1 mM bMe and then resuspended in 10 mL 25 mM HEPES pH 7.5,100 mM KCl, 10% glycerol, 1% w/v OGP, 1 mM bMe and cleaved with thrombin over night at 4° C., and further for 8 hours at room temperature. 10 mM imidazole was added to the beads and incubated for 30 min at room temperature. Supernatant was concentrated and beads were washed with 25 mM HEPES pH 7.5,100 mM KCl, 10% glycerol, 1% w/v OGP, 1 mM bMe. The supernatant (50 ml) was pooled and concentrated, and the protein was aliquoted and frozen.

The SNARE domain of rat synaptobrevin (amino acids 49-96) fused to eGFP (SybSN-GFP) was expressed as His₆-tagged fusion protein. Cells were grown at 37° C. until OD₆₀₀ of 0.3-0.5, induced with 40 μM IPTG, and grown overnight at 18° C. Cells were harvested and resuspended in 25 mM HEPES pH 7.5, 400 mM KCl, 5% Triton X-I00, 2 mM MgCl₂, 1 mM bMe, EDTA-free Complete protease inhibitors (Roche), and DNaseI. Cells were lysed by freeze thawing and the lysate was centrifuged for 45 min at 20,000× g, 4° C. Supernatant was incubated with 0.5 mL of Ni-NTA agarose beads (Qiagen) per 1 L of culture for 2 h. Beads were washed 7 times with 25 mM HEPES pH 7.5, 400 mM KCl, 1 mM bMe. 3 more wash steps were conducted with increasing concentrations of imidazole up to 25 mM. Protein was eluted with 150 mM imidazole; dialyzed into 25 mM HEPES pH 7.5, 100 mM KCl, 2 mM DTT; aliquoted and frozen.

The rat Doc2b (NP_{—}112404) C2AB domain fragment (amino acids 125-412) was expressed as a GST fusion protein in BL21 (DE3) pLysS cells. Cells were grown at 37° C. until OD₆₀₀ of 0.3, induced with 40 μM IPTG and grown for 14-16 h at 18° C. Cells were harvested and resuspended in 50 mM HEPES pH 7.5, 300 mM NaCl, 4 mM DTT, 2 mM MgCl₂, DNaseI, RNaseA, EDTA-free Complete protease inhibitors (Roche) and lysed by freeze thawing. The lysate was centrifuged for 45 min at 125,000× g, 4° C., and the supernatant was incubated with 1 mL of glutathione-sepharose beads per 1 L of culture for 1-2 h. Beads were washed 7 times with 50 mM HEPES pH 7.5, 300 mM NaCl, 4 mM DTT followed by two 15-min washes with 50 mM HEPES pH 7.5, 500 mM NaCl, 4 mM DTT, 2 mM MgCl₂, DNaseI, RNaseA. The protein was cleaved off the beads with thrombin by overnight incubation at 16° C. The supernatant was concentrated and the protein was further purified by gel filtration in 50 mM HEPES pH 7.5, 150 mM NaCl, 4 mM DTT using a HiLoad 16/60 Superdex 75 column (Pharmacia Biotech). The protein was concentrated again, flash frozen and stored at −80° C.

Rat full length synaptobrevin (NP_{—}036795, Syb), full-length SNAP25b (NP_{—}112253, SNAP25) and a syntaxinla (NP_{—}446240) construct (amino acids 180-288) that lacks the N-terminal regulatory domain, which is composed of the helices Ha, Hb and Hc (syntaxinlaΔHabc), were expressed as GST fusion proteins. Expression was conducted as described previously. The protocol for syntaxinlaΔHabc purifcation was the same as for SNAP25.

Possible protein and RNA contamination were checked by SDS gel electrophoresis and UV spectroscopy, respectively. The functionality of SNARE proteins was monitored by SDS-resistant SNARE complex formation (data not shown).

Proteo-liposome preparation. The GFP-Syb, tSNARE and vSNARE liposomes were prepared by detergent assisted insertion into pre-formed liposomes.

GFP-Syb liposomes: 1,2-diphytanoyl-sn-glycero-3-phosphocholine (DPhPC) (Avanti) lipids in chloroform were dried under nitrogen and desiccated for 90 min. Lipids were rehydrated by the addition of 50 mM Tris pH 8, 150 mM NaCl, 2 mM DTT. Lipids were incubated in the buffer for 15 min at room temperature and tip sonicated to create a 10 mM liposome solution. Solution was spun for 20 min at 10,000× g at room temperature to spin out aggregates. 20 μL of the liposomes were then added to 80 μL of a 10 μM solution of GFP-Syb and incubated for 15 min at room temperature. The detergent was then diluted below the critical micelle concentrationby the addition of 100 μL 50 mM Tris pH 8, 150 mM NaCl, 2 mM DTT. The liposomes were then dialyzed against 2L of 25 mM HEPES pH 7.5,100 mM KCl, 2 mM DTT, 10 g BioBeads (BioRad) over night at 4° C. to remove the detergent and spun at 10,000× g for 5 min at room temperature to remove aggregates. The supernatant was used for experiments.

tSNARE liposomes: 1,2-diphytanoyl-sn-glycero-3-phosphocholine (DPhPC) (Avanti) lipids in chloroform were dried under nitrogen and desiccated for 90 min. Lipids were rehydrated by the addition of 50 mM Tris pH 8, 150 mM NaCl, 2 mM DTT. Lipids were incubated in the buffer for 15 min at room temperature and tip sonicated to create a 20 mM liposome solution. Solution was spun for 20 min at 10,000× g at room temperature to spin out aggregates. 10 μL of the liposomes were then added to 90 μL of an 11.1 μM preformed tSNARE complex solution (1:1 SNAP25 and syntaxinlaΔHabc, 1 hr, room temperature) and incubated for 15 min at room temperature. The detergent was then diluted below the critical micelle concentration by the addition of 100 μL 50 mM Tris pH 8, 150 mM NaCl, 2 mM DTT. The liposomes were then dialyzed overnight against 2L of 25 mM HEPES pH 7.5,100 mM KCl, 2 mM DTT, 10 g BioBeads (BioRad) at 4° C. to remove the detergent and spun at 10,000× g for 5 min at room temperature to remove aggregates. The supernatant was used for experiments.

vSNARE liposomes: To create a 10 mM liposome suspension, lipids stored in chloroform were mixed in the following ratio: 5% 1,2-diphytanoyl-sn-glycero-3-phospho-L-serine (DPhPS) (Avanti), 10% cholesterol (Avanti), 65% 1,2-diphytanoyl-sn-glycero-3-phosphocholine(DPhPC) (Avanti), 20% 1,2-diphytanoyl-sn-glycero-3-phosphoethanolamine (DPPE) (Avanti); dried under nitrogen; and desiccated for 90 μM. Lipids were rehydrated by the addition of 50 mM Tris pH 8, 150 mM NaCl, 2 mM DTT. Lipids were incubated in the buffer for 15 min at room temperature and tip sonicated. Solution was spun for 20 min at 10,000× g at room temperature tospin out aggregates. 20 μL of the liposomes were then added to 80 μL of a 10 μM premixed solution of Syb and GFP-Syb and incubated for 15 min at room temperature. The detergent was then diluted below the critical micelle concentration by the addition of 100 μL 50 mM Tris pH 8, 150 mM NaCl, 2 mM DTT. The liposomes were dialyzed overnight against 2 L of 25 mM HEPES pH 7.5, 100 mM KCl, 2 mM DTT, 10 g BioBeads (BioRad) at 4° C. to remove the detergent and spun at 10,000× g for 5 min at room temperature to remove aggregates. The supernatant was used for experiments.

Preparation of “supplement” liposomes. Supplement liposomes, which were used to control lipid composition independently from tSNARE concentration in SUV-GUV fusion experiments, were prepared by the method of sonication. Lipids stored in chloroform were mixed to the following ratio: 70% 1,2-diphytanoyl-sn-glycero-3-phosphocholine (DPhPC) (Avanti), 20% 1,2-diphytanoyl-sn-glycero-3-phospho-L-serine (DPhPS) (Avanti), 10% cholesterol (Avanti); dried under nitrogen; and desiccated for 90 min. Lipids were rehydrated to a final concentration of 1 mg/ml by the addition of 50 mM Tris pH 8, 150 mM NaCl, 2 mM DTT KCl for 15 min, briefly vortexed, and then tip sonicated. Solution was spun for 20 min at 10,000× g at room temperature and the supernatant was used for experiments.

Chamber design. Chambers were typically designed with two cylindrical bores (4 mm or 6 mm diameter) separated by a 0.5 mm-wide slot through the center, forming a 3 mm-wide window between the two cylinders (FIG. 11a). Chambers were cut from sheets of 3 mm- or 4.5 mm-thick acrylic (McMaster-Carr) using a laser cutter (Versa Laser). A 1.5 mm hole was drilled in one end of the chamber, for insertion and alignment of the inkjet nozzle. This hole was covered by a 0.26 mm latex film (McMaster-Carr), forming a seal in which a small hole was made with a 23G needle. The chamber was then cemented (acrylic cement, TAP plastics) to a 0.2 mm thick acrylic bottom (Astra products) to facilitate imaging with short working distance objective lenses. Chambers were cleaned with 2% Neutrad (Decon Laboratories) solution at 60° C., thoroughly rinsed and re-used multiple times.

Planar bilayer formation. A planar bilayer was typically prepared by incubating two SUV containing droplets, separated by a thin acrylic divider, in a decane-loaded acrylic chamber. We optimized the incubation time to ensure adequate transfer o flipid to the oil-water interface. Fusion of SUVs to a hydrophobic surface is a diffusion limited process. Thus, we optimized incubation time based on a simple random walk model that calculated the distribution of arrival times for SUVs to reach the oil-water interface by diffusion. We found that ˜90% of 25 nm diameter SUV s (average size by tip sonication) which were initially uniformly distributed in a 4 mm diameter spherical water droplet would reach the surface of the droplet within 12 hours, and so incubation was typically done overnight at 4° C. However, when forming asymmetric bilayers, the incubation time was reduced to tens of minutes to prevent trans-bilayer mixing of lipids by diffusion through the oil. Liposome concentrations in the droplets typically ranged from 0.05-0.2 mg/ml. If 0.05 mg/ml or lower SUVs were used, DPhPC was added to the decane to a final concentration of ˜20 mg/ml shortly before bilaye rformation to improve stability of the bilayer. This was unnecessary when higher SUV concentrations were used. Bilayer formation was initiated by removal of the thin acrylic divider.

Vesicle formation by microfluidic jetting. GUVs were formed by microfluidic jetting against planar bilayer substrates. A micro-nozzle was formed by pulling a glass capillary (0.6 mm ID, 0.75 mm OD) to a fine tip using a P-97 Micropipette Puller (Sutter Instruments), and then subsequently melting and sanding the tip until the orifice was 10-15 μm ID. Micro-nozzles were mounted into piezo-electric inkjet devices by MicroFabTechnologies, and are referred to as inkjets. Contents to be encapsulated in GUVs were loaded into a disposable syringe, and back-filled into an inkjet. The loaded inkjet was inserted into a custom chamber containing a pre-formed bilayer, and aligned within 200 μm from the planar bilayer. A microfluidic jet was formed from multiple pulses of the piezo-electric inkjet, and the pulse train was controlled by the drive electronics (MicroJet III controller box, MicroFabTechnologies). Typical shot profiles were 15-35 identical trapezoidal pulses repeated at 20 kHz with 25-35V amplitude, and 3 μs rise time, 35 μs duration and 3 μs fall time. A wide range of solutions were encapsulated in GUVs using this technique, but the encapsulated solution wasalways osmotically balanced to the inner and outer droplet solutions, and typically contained >100 mOsm sucrose to sink the GUV for ease of imaging. Vesicle formation was done on an inverted microscope (Axiovert 200, Zeiss) and recorded using a high-speed camera (1024PCI, Photron).

Imaging and analysis. GUVs were transferred, within the acrylic chamber, to a neighboring microscope (AxioObserver, Zeiss) for confocal microscopy with a cooled EMCCD camera (Cascade II, Roper Scientific). Images were analyzed using ImageJ (developed by the National Institutes of Health) and MatLab (Mathworks). Particle tracking was accomplished using automated tracking software adapted for use in MatLab (http://www. physics.georgetown.edu/matlab/).

### Example 4

### Incorporation of Transmembrane Proteins With Control of Orientation Additional Experimental Details

Proteins, planar lipid membranes, SUVs, and GUVs were prepared as described in Example 3 with the modifications and additions that follow. GFP-Syb was incorporated into GUV s with the GFP domain facing outwards. A thin acrylic divider was placed in a 4 5 mm-deep acrylic chamber with 4 mm diameter bores and the chamber was loaded with 40 μL decane. 45 μL of 0.05 mg/ml GFP-Syb liposomes was added to the outer droplet, 45 μL of 0.05 mg/ml DPhPC liposomes was added to the inner droplet, and the chamber was incubated overnight at 4° C. Roughly one hour before the experiment, DPhPC in decane was added to a final concentration of 20 mg/ml in the chamber. The thin acrylic divider was removed and an inkjet was loaded with 6% iodixanol, 25 mM HEPES pH 7.5, 100 mM KCl, and 2 mM DTT to form GUVs. After formation of multiple GUVs from a single bilayer, images of the GUVs were captured using confocal microscopy. Protease K (Sigma-Aldrich) was added to the outer droplet to a final concentration of 0.2 mg/mL and mixed. A second set of images were captured of all GUVs after Protease K addition. The average membrane intensity was calculated for each vesicle, before and after exposure to Protease K by thresholding the raw images. Membrane intensity was calculated by subtracting the average of the dimmest 90% of the image pixels (background) from the average of the brightest 1% of the image pixels (vesicle). Each thresholded image was checked visually for agreement with the raw image. The fraction of GFP-Syb with the GFP domain oriented outwards was calculated from the membrane intensity before and after addition of Protease K.

Likewise, GFP-Syb was incorporated into GUVs with the GFP domain facing inwards by loading 45 μL of 0.05 mg/mL GFP-Syb liposomes into the inner droplet, and 45 μL of 0.05 mg/mL DPhPC liposomes to the outer droplet for overnight incubation. GUVs with GFP-Syb oriented symmetrically were formed by incubating 45 μL of 0.05 mg/ml GFP-Syb liposomes in both droplets. In both cases, analysis of GFP-Syb orientation was conducted as described above.

### Results and Discussion

As a next step in creating cell-like vesicles, we incorporated and oriented transmembrane proteins into GUV membranes through an extension of the SUV fusion method. We used the well-characterized SNARE protein synaptobrevin (Syb), which has a single membrane-spanning a-helical domain. A GFP fusion of synaptobrevin (GFP-Syb) was first incorporated into SUVs by the standard protocol of detergent assisted insertion. Planar bilayers were formed from droplets containing SUVs with GFP-Syb, and GUVs with GFP-Syb were made by microfluidic jetting. The GUVs displayed bright, uniform fluorescence along their membrane, suggesting successful incorporation of the transmembrane protein (FIG. 13a).

As a second example of protein insertion into membranes, we incorporated the nonfluourescent transmembrane protein syntaxin (syntaxinlaΔHabc) into GUVs using the same approach. We confirmed membrane localization of the syntaxin and tested its functionality by forming SNARE complexes containing SNAP25 and a soluble version of synaptobrevin fused to GFP (SybSN-GFP) (FIG. 13b). Strong localization of SybSN-GFP to the membrane indicates that functional syntaxin was successfully incorporated into the membrane.

Synaptobrevin is normally found in cell membranes with its hydrophilic SNARE domain oriented into the cytosol and its hydrophobic transmembrane domain inserted in the bilayer. To emulate this geometry and protein orientation, we incubated GFP-Syb SUVs in the inner droplet (FIG. 13c), where the microfluidic jet is inserted to form GUVs, and protein-free SUVs in the other droplet. Orientation was measured by a fluorescence protease protection assay that works by probing the accessibility of a fluorescently-labeled domain of a transmembrane protein to a membrane-impermeable protease added to the external solution. We conducted this assay for GUVs formed from planar bilayers made by incubating GFP-Syb SUVs in three different configurations: (i) only the outer droplet (FIG. 13c, left column), (ii) both droplets (FIG. 13c, middle column), or (iii) only the inner droplet (FIG. 13c, right column) The fraction of GFP-Syb with a given orientation was calculated from the ratio of the fluorescence intensity, measured by fluorescence microscopy, before and after addition of Protease K. Quantification of this data revealed that in configuration (i) 90+/−2% (s.e.m., n=6 bilayers), in (ii) 51+/−3% (s.e.m., n=4 bilayers) and in (iii) 9+/−5% (s.e.m., n=4 bilayers) of GFP-Syb was oriented outwards, therefore confirming control over the orientation of incorporated transmembrane protein in GUVs (FIG. 13d). GFP-Syb orientation was stable over many hours.

### Example 5

### Reconstitution of SNARE-Mediated Membrane Fusion in GUVs

### Additional Experimental Details

Proteins, planar lipid membranes, SUVs, and GUVs were prepared as described in Example 3 with the modifications and additions that follow. Planar bilayers containing tSNAREs were pre-formed by overnight incubation. A thin acrylic divider was placed in a 4.5 mm-deep acrylic chamber with 4 mm diameter bores and the chamber was loaded with 40 μL decane (Sigma-Aldrich). 45 μL droplets containing 0.05 mg/mL tSNARE liposomes and 0.05 mg/mL supplement SUVs in 25 mM HEPES pH 7.5,100 mM KCl, 0.4 mM CaCl₂, 2 mM DTT were added to both sides of the chamber, and the chamber was incubated overnight at 4° C. Based on the assumption of equal transfer of tSNARE SUVs and supplement SUVs to the oil-water interface, we estimated the final lipid composition in the tSNARE GUVs to be 85% DPhPC, 10% DPhPS and 5% cholesterol. Roughly one hour before the experiment, DPhPC in decane was added to a final concentration of 20 mg/ml in the chamber. The thin acrylic divider was removed, forming a tSNARE-containing planar bilayer, and an inkjet loaded with 2 μg/mL vSNARE liposomes, 0.5 μM Doc2, 6% iodixanol, 25 mM HEPES pH 7.5,100 mM KCl, and 2 mM DTT was used to form GUVs. Typically 3-5 GUVs were formed from a bilayer, and the best GUV was identified and tracked by confocal microscopy for up to an hour. The internal volume of a GUV was observed by capturing a time series of z-stacks, with images taken every 5 μm in height over 50-100 μm, and full z-stacks taken every 10-15 s.

### Results and Discussion

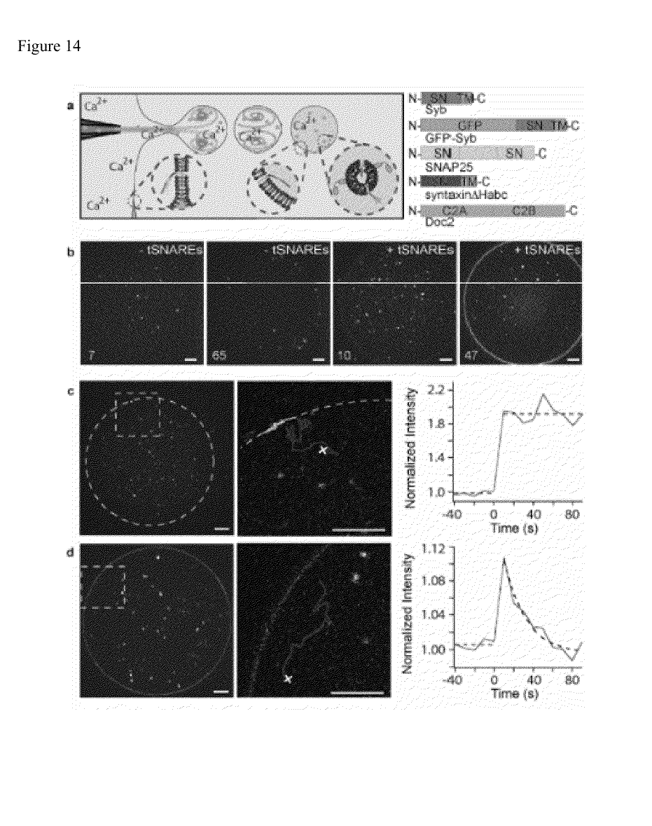

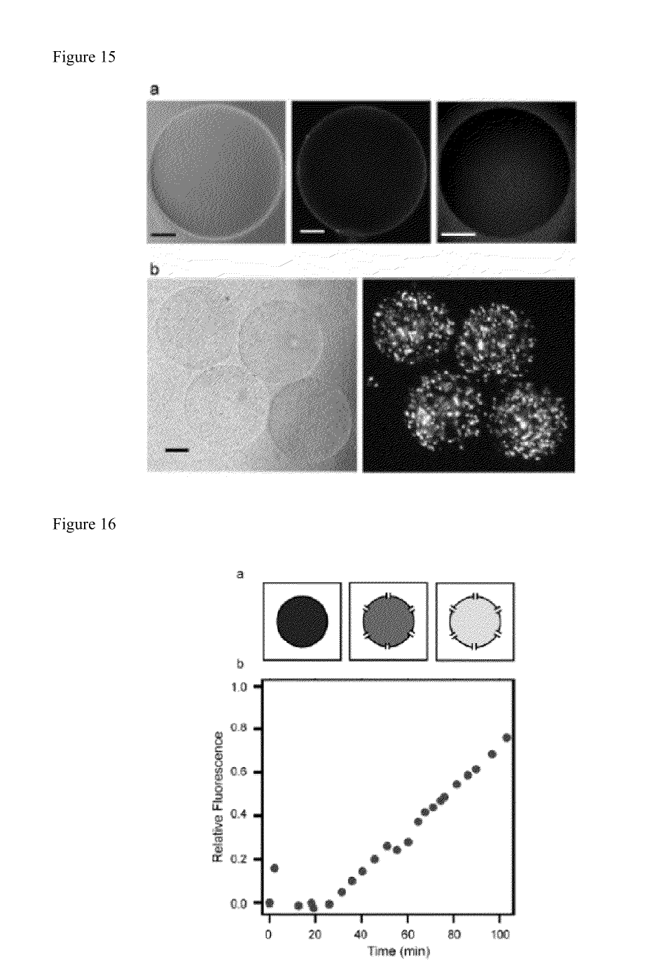

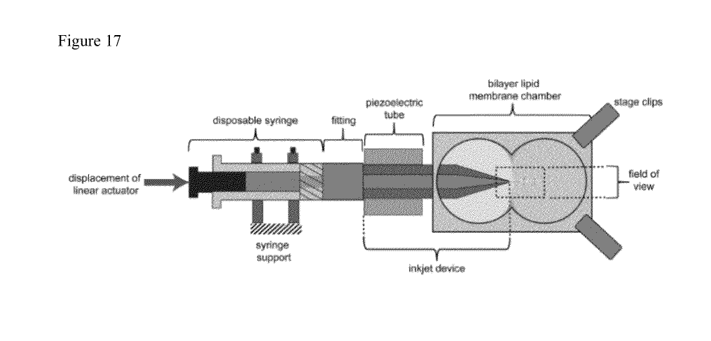

Thus far, we have demonstrated the ability to form GUV s with controlled lipid composition, bilayer asymmetry, and oriented transmembrane proteins. To test the utility of this technique for cellular reconstitutions that depend on membrane composition and defined internal contents, we reconstituted SNARE-mediated membrane fusion within a giant vesicle in a configuration that closely mimics the physiological geometry. Our assay was based on vSNARE SUVs (65% DPhPC, 20% DPPE, 15% DPhPS, 10% Cholesterol, Syb/GFP-Syb) loaded into tSNARE-GUVs (85% DPhPC, 10% DPhPS, 5% Cholesterol, SNAP25/syntaxinlaΔHabc). To increase fusion efficiency we co-encapsulated the synaptotagmin-related protein domain Doc2bC2AB (Doc2) with vSNARE-SUVs into the tSNARE-GUVs. Giant vesicles containing the tSNARE proteins were formed by microfluidic jetting in a manner similar to the experiments above. An inkjet nozzle was loaded with a controlled ratio of vSNARE SUVs and Doc2 (lipid:Syb:Doc2=250:1:60), and this solution was jetted into tSNARE-GUVs by deformation of a tSNARE-containing planar bilayer (FIG. 14a). We added CaCl₂ (400 μM) to the aqueous droplets surrounding the planar membrane, but not to the inkjet solution, so that when we formed GUVs, entrainment of CaCl₂ from the surrounding fluid initiated the activity of Doc2 at the exact moment of encapsulation within the GUV. Control experiments were performed with GUVs containing only syntaxin and protein-free GUVs.

We observed diffusion of green fluorescent punctae (GFP-Syb in clustered SUVs) within the GUVs by time-lapse fluorescence microscopy. At each time point, three dimensional images of the vesicle volume were captured by taking multiple cross-sectional images throughout a 50-100 μm vertical window in 5 μm increments. Full three-dimensional images were captured every 10 s, yielding the time evolution of the population of punctae diffusing through the closed volume of the GUV.

Several observations could be made from watching the SUVs diffuse within the GUVs. First, we found that addition of Doc2 caused vSNARE-SUVs to form slowly diffusing clusters (D<1 μm2/s). Without being bound to any particular theory, we suspect this is due to the two linked C2 domains binding to two apposing SUV membranes. Secondly, we noted that Doc2 was able to dock SUV s clusters to GUV membranes,even in the absence of tSNAREs (FIG. 14b, -tSNAREs). Finally, when GUVs were made containing tSNAREs, we observed fewer vSNARE-SUV s docked at the tSNARE-GUV membrane but found that the overall GUV membrane increased in fluorescence over time, suggesting that GFP-Syb was transferred to the GUV membrane via liposome fusion (FIG. 14b,+tSNAREs).

To obtain a more detailed picture of what happened to SUVs confined within the GUVs,we used automated tracking software to follow the location of individual punctae over the course of the experiment. We observed two classes of events: docking and fusion. In the case of docking, SUV clusters contacted the inner leaflet of the GUV membrane, and their diffusional motion was abruptly confined to the surface of the GUV (FIG. 14c). After docking, the SUV clusterremained bound to the GUV membrane and slowly diffused along its surface. We calculated themembrane intensity at the docking site during the docking event (FIG. 14c) and saw that thefluorescence intensity spiked at the site of the docking event indicating arrival of the SUV clusterat the membrane. Over time, the fluorescent intensity persisted in magnitude for the duration ofdata acquisition (4 min), indicating stable docking.

In the case of fusion, we observed SUV clusters contacting the inner leaflet of the GUV membrane and immediately disappearing from sight (FIG. 14d). This is believed to result from the rapid dilution of the punctate SUV fluorescence into the large continuous membrane of the GUV, contributing to the overall increase in membrane fluorescence previously described. We confirmed that these events were not due to vertical diffusion out of the observed volume by tracking the vertical position of these punctae immediately prior to the fusion event. To follow this event, we again calculated the membrane intensity at the contact site (FIG. 14d). In contrast to docking, the local fluorescence intensity spike at the membrane was not persistent but rather steadily decayed to the baseline value, indicating that SUV-GUV fusion occurred. In control experiments where we do not incorporate tSNAREs into the GUV membrane, we observe only docking but not fusion (10 independent experiments).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A vesicle composition comprising:
a unilamellar bilayer comprising a bilayer lipid and at least one bilayer component each independently selected from the group consisting of a membrane protein and a functionalized lipid; and
a component encapsulated by the unilamellar bilayer, wherein the encapsulated component is selected from the group consisting of a protein, a peptide, an enzyme, an oligonucleotide, and a polynucleotide.

2. The vesicle composition of claim 1, wherein the bilayer lipid is 1,2-diphytanoyl-sn-glycero-3-phosphocholine (DPhPC).

3. The vesicle composition of claim 1, wherein the functionalized lipid is selected from the group consisting of a PEGylated lipid, a signaling lipid, and a chelating lipid.

4. The vesicle composition of claim 3, wherein the functionalized lipid is selected from the group consisting of a PEGylated lipid, tetramethylrhodamine-phosphatidylinositol(4,5)-bisphosphate (TMR-PIP₂), and (1,2-dioleoyl-sn-glycero-3-[(N-(5-amino-1-carboxypentyl)-iminodiacetic acid)succinyl]) (DOGS-NTA).

5. The vesicle composition of claim 1, wherein the bilayer component is the membrane protein.

6. The vesicle composition of claim 5, wherein the membrane protein is soluble N-ethylmaleimide-sensitive factor attachment protein receptor (SNARE) protein synaptobrevin.

7. The vesicle composition of claim 5, wherein the vesicle further comprises an interior and an exterior, such that more than 50% of the membrane protein is oriented towards either the interior or exterior of the vesicle.

8. The vesicle composition of claim 1, wherein the unilamellar bilayer further comprises an inner leaflet and an outer leaflet, wherein
the outer leaflet comprises the bilayer lipid and the functionalized lipid; and
the inner leaflet comprises the bilayer lipid, thereby forming an asymmetric unilamellar bilayer.

9. The vesicle composition of claim 1, wherein the unilamellar bilayer further comprises an inner leaflet and an outer leaflet, wherein
the outer leaflet comprises the bilayer lipid; and
the inner leaflet comprises the bilayer lipid and the functionalized lipid, thereby forming an asymmetric unilamellar bilayer.

10. The vesicle composition of claim 1, wherein the unilamellar bilayer encapsulates a second component selected from the group consisting of a protein, a peptide, an enzyme, an oligonucleotide, a polynucleotide, a diagnostic agent and a therapeutic agent.

11. The vesicle composition of claim 1, wherein the unilamellar bilayer further comprises an inner leaflet and an outer leaflet, and wherein the unilamellar bilayer comprises
diphytanoylphosphatidylcholine (DPhPC) and tetramethylrhodamine-phosphatidylinositol(4,5)-bisphosphate (TMR-PIP₂), or
DPhPC and (1,2-dioleoyl-sn-glycero-3-[(N-(5-amino-1-carboxypentyl)-iminodiacetic acid)succinyl])-Ni (DOGS-NTA-Ni), such that the outer leaflet comprises DPhPC and the inner leaflet comprises DPhPC and DOGS-NTA-Ni, and optionally encapsulating His-green fluorescent protein, or
DPhPC and DOGS-NTA-Ni, such that the outer leaflet comprises DPhPC and DOGS-NTA-Ni, and the inner leaflet comprises DPhPC, encapsulating iodixanol and optionally His-green fluorescent protein, or
DPhPC, diphytanoylphosphatidylserine (DPhPS), cholesterol, and the membrane protein trans-soluble N-ethylmaleimide-sensitive factor attachment protein receptor (tSNARE), encapsulating SybSN-GFP and iodixanol, or
DPhPC, DPhPS, cholesterol, and the membrane protein tSNARE, encapsulating iodixanol, and functionalized on the outer leaflet with SybSN-GFP.

12. The vesicle composition of claim 1, wherein the vesicle has a diameter of from about 0.1 μm to about 5 mm.

13. A method of forming a vesicle, the method comprising:
contacting an aqueous mixture and an oil mixture, wherein the aqueous mixture comprises a first lipid, and the oil mixture comprises a second lipid, wherein
the aqueous mixture or oil mixture further comprises at least one bilayer component each selected from the group consisting of a transmembrane protein and a functionalized lipid,
such that a lipid bilayer forms at the interface of the aqueous mixture and the oil mixture,
wherein the interfacial lipid bilayer comprises an aqueous mixture lipid layer comprising the first lipid, and an oil mixture lipid layer comprising the second lipid,
wherein the interfacial lipid bilayer further comprises the membrane protein and the functionalized lipid when present;
pulsing the interfacial lipid bilayer with a fluid mixture from an inkjet, wherein the fluid mixture comprises at least one component selected from the group consisting of a protein, a peptide, an enzyme, an oligonucleotide, and a polynucleotide,
thereby forming the vesicle.

14. The method of claim 13, wherein the first and second lipids are the same, the aqueous mixture further comprises the functionalized lipid, and wherein
the aqueous mixture lipid layer comprises the first lipid and the functionalized lipid; and
the oil mixture lipid layer comprises the second lipid, thereby forming an asymmetric interfacial lipid bilayer.

15. The method of claim 13, wherein the first and second lipids are the same and the oil mixture further comprises the functionalized lipid, and wherein
the aqueous mixture lipid layer comprises the first lipid; and
the oil mixture lipid layer comprises the second lipid and the functionalized lipid, thereby forming an asymmetric interfacial lipid bilayer.

16. The method of claim 13, wherein the first and second lipids are the same, and the aqueous mixture or the oil mixture further comprises the membrane protein.

17. The method of claim 13, wherein the fluid mixture further comprises a second component selected from the group consisting of a protein, a peptide, an enzyme, an oligonucleotide, a polynucleotide, a diagnostic agent and a therapeutic agent.
